SEQUENCE LISTING (65 sequences)

SEQ ID NO: 1 - DNA, 1653 bp, Photinus pyralis
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 2 - protein, 19 aa, Simian virus 40
LMDPKKKRKVDPKKKRKVG

SEQ ID NO: 3 - DNA, 593 bp, Artificial
  internal ribosome entry site in pIRES2-EGFP
gatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 4 - protein, 15 aa, Artificial
  Linker
GGGGSGGGGSGGGGS

SEQ ID NO: 5 - protein, 15 aa, Artificial
  Linker
EAAAREAAAREAAAR

SEQ ID NO: 6 - DNA, 25 bp, Artificial
  Primer
atggaagatgccaaaaacattaaga

SEQ ID NO: 7 - DNA, 25 bp, Artificial
  Primer
ttacacggcgatcttgccgcccttc

SEQ ID NO: 8 - DNA, 25 bp, Artificial
  Primer
tacaaacgctctcatcgacaaggac

SEQ ID NO: 9 - DNA, 25 bp, Artificial
  primer
gtccttgtcgatgagagcgtttgta

SEQ ID NO: 10 - DNA, 25 bp, Artificial
  Primer
ggctggctgcacagcggcgacatcg

SEQ ID NO: 11 - DNA, 25 bp, Artificial
  Primer
gaagggcggcaagatcgccgtgtaa

SEQ ID NO: 12 - DNA, 25 bp, Artificial
  Primer
atggatgacgatatcgctgcgctgg

SEQ ID NO: 13 - DNA, 25 bp, Artificial
  primer
catcgtgcaccgcaagtgcttctag

SEQ ID NO: 14 - DNA, 25 bp, Artificial
  Primer
ctagaagcacttgcggtgcacgatg

SEQ ID NO: 15 - DNA, 25 bp, Artificial
  Primer
cagatttcaactattgcagaaagtg

SEQ ID NO: 16 - DNA, 25 bp, Artificial
  Primer
agaagaaaagtcagaagaggagact

SEQ ID NO: 17 - DNA, 25 bp, Artificial
  Primer
agtctcctcttctgacttttcttct

SEQ ID NO: 18 - DNA, 25 bp, Artificial
  Primer
ggtgttcgaaaaggctggcatgaac

SEQ ID NO: 19 - DNA, 25 bp, Artificial
  Primer
taaaatacaaaaagaactagaagaa

SEQ ID NO: 20 - DNA, 25 bp, Artificial
  Primer
ttcttctagttctttttgtatttta

SEQ ID NO: 21 - DNA, 57 bp, Artificial
  Nuclear Localization Signal
cttatggatccaaaaaagaagagaaaggtagaccctaagaaaaagaggaaagttggg

SEQ ID NO: 22 - DNA, 25 bp, Artificial
  Primer
cttatggatccaaaaaagaagagaa

SEQ ID NO: 23 - DNA, 25 bp, Artificial
  Primer
ccctaagaaaaagaggaaagttggg

SEQ ID NO: 24 - DNA, 25 bp, Artificial
  Primer
cccaactttcctctttttcttaggg

SEQ ID NO: 25 - DNA, 22 bp, Artificial
  Primer
gatccgcccctctccctccccc

SEQ ID NO: 26 - DNA, 24 bp, Artificial
  Primer
ggttgtggccatattatcatcgtg

SEQ ID NO: 27 - DNA, 45 bp, Artificial
  Linker
ggaggtgggggtagtgggggcggaggtagcggtggcggtggtagt

SEQ ID NO: 28 - DNA, 21 bp, Artificial
  Primer
ggaggtgggggtagtgggggc

SEQ ID NO: 29 - DNA, 21 bp, Artificial
  Primer
actaccaccgccaccgctacc

SEQ ID NO: 30 - DNA, 25 bp, Artificial
  Primer
gcgcagcaccatggcctgaaataac

SEQ ID NO: 31 - DNA, 25 bp, Artificial
  Primer
aggcctaggcttttgcaaaaagctt

SEQ ID NO: 32 - DNA, 25 bp, Artificial
  Primer
aagctttttgcaaaagcctaggcct

SEQ ID NO: 33 - DNA, 47 bp, Artificial
  Primer
cgcccccactacccccacctccgtccttgtcgatgagagcgtttgta

SEQ ID NO: 34 - DNA, 48 bp, Artificial
  Primer
gaggtagcggtggcggtggtagtatggatgacgatatcgctgcgctgg

SEQ ID NO: 35 - DNA, 44 bp, Artificial
  Primer
cgcccccactacccccacctccgaagggcggcaagatcgccgtg

SEQ ID NO: 36 - DNA, 37 bp, Artificial
  Primer
actacccccacctccgaagcacttgcggtgcacgatg

SEQ ID NO: 37 - DNA, 39 bp, Artificial
  Primer
ggtggcggtggtagtaagggtgggcgcgccgagccagct

SEQ ID NO: 38 - DNA, 22 bp, Artificial
  Primer
gaagcacttgcggtgcacgatg

SEQ ID NO: 39 - DNA, 25 bp, Artificial
  Primer
atggaagatgccaaaaacattaaga

SEQ ID NO: 40 - DNA, 25 bp, Artificial
  Primer
ggctggctgcacagcggcgacatcg

SEQ ID NO: 41 - DNA, 45 bp, Artificial
  Primer
ccgcccccactacccccacctccgaagcacttgcggtgcacgatg

SEQ ID NO: 42 - DNA, 47 bp, Artificial
  Primer
aggtagcggtggcggtggtagtggctggctgcacagcggcgacatcg

SEQ ID NO: 43 - DNA, 47 bp, Artificial
  Primer
cgcccccactacccccacctccagtctcctcttctgacttttcttct

SEQ ID NO: 44 - DNA, 47 bp, Artificial
  Primer
gaggtagcggtggcggtggtagtatggaagatgccaaaaacattaag

SEQ ID NO: 45 - DNA, 48 bp, Artificial
  Primer
gaggtagcggtggcggtggtagtggctggctgcacagcggcgacatcg

SEQ ID NO: 46 - DNA, 47 bp, Artificial
  Primer
cgcccccactacccccacctccttcttctagttctttttgtatttta

SEQ ID NO: 47 - DNA, 47 bp, Artificial; identical to SEQ ID NO: 44
  Primer

SEQ ID NO: 48 - DNA, 48 bp, Artificial; identical to SEQ ID NO: 45
  Primer

SEQ ID NO: 49 - DNA, 40 bp, Artificial
  Primer
actacccccacctccgtccttgtcgatgagagcgtttgta

SEQ ID NO: 50 - DNA, 39 bp, Artificial; identical to SEQ ID NO: 37
  Primer

SEQ ID NO: 51 - DNA, 37 bp, Artificial
  Primer
actacccccacctcccacggcgatcttgccgcccttc

SEQ ID NO: 52 - DNA, 22 bp, Artificial
  Primer
cacggcgatcttgccgcccttc

SEQ ID NO: 53 - DNA, 48 bp, Artificial
  Primer
gaggtagcggtggcggtggtagtcagatttcaactattgcagaaagtg

SEQ ID NO: 54 - DNA, 44 bp, Artificial
  Primer
cgcccccactacccccacctcccacggcgatcttgccgcccttc

SEQ ID NO: 55 - DNA, 48 bp, Artificial
  Primer
gaggtagcggtggcggtggtagtggtgttcgaaaaggctggcatgaac

SEQ ID NO: 56 - DNA, 25 bp, Artificial
  Primer
tgcctacaggaaaattttgaatgac

SEQ ID NO: 57 - DNA, 25 bp, Artificial
  Primer
ggcctccttgaaaggatttcccttc

SEQ ID NO: 58 - protein, 14 aa, Artificial
  Amino acid sequence added by Multisite Gateway System
KGGRADPAFLYKVE

SEQ ID NO: 59 - DNA, 28 bp, Artificial
  Primer
ggggacaactttgtatagaaaagttgaa

SEQ ID NO: 60 - DNA, 27 bp, Artificial
  Primer
ggggactgcttttttgtacaaacttga

SEQ ID NO: 61 - DNA, 31 bp, Artificial
  Primer
ggggacaagtttgtacaaaaaagcaggcttt

SEQ ID NO: 62 - DNA, 30 bp, Artificial
  Primer
ggggaccactttgtacaagaaagctgggtt

SEQ ID NO: 63 - DNA, 28 bp, Artificial
  Primer
ggggacagctttcttgtacaaagtggaa

SEQ ID NO: 64 - DNA, 27 bp, Artificial
  Primer
ggggacaactttgtataataaagttgt

SEQ ID NO: 65 - protein, 24 aa, Artificial
  Linker
GGGGSGGGGSKGGRADPAFLYKVE

(19) United States
(12) Patent Application Publication
(10) Pub. No.: US 2011/0154513 A1
(43) Pub. Date: Jun. 23, 2011

(54) PROBE FOR VISUALIZING NEURAL ACTIVITY

(75) Inventors: Tetsuya Ishimoto, Toyama (JP); Hisashi Mori, Toyama (JP); Hironori Izumi, Toyama (JP)

(73) Assignee: University of Toyama, Toyama-shi (JP)

(21) Appl. No.: 13/058,562

(22) PCT Filed: Aug. 12, 2009
(86) PCT No.: PCT/JP2009/064225

§ 371 Date: Feb. 11, 2011

(30) Foreign Application Priority Data
Aug. 12, 2008 (JP) 2008-207546
Feb. 5, 2009 (JP) 2009-024382

(51) Int. Cl. C12Q 1/66; C12N 9/02; C12N 15/53; A01K 67/027; A61K 49/00

(52) U.S. Cl. 800/3; 435/189; 536/23.2; 435/8; 800/14

(57) ABSTRACT

An object of the present invention is to develop a probe for measuring in real time the kinetics of CREB or actin closely related to brain functions such as memory formation in live animals. The probe used in the present invention is a probe comprising luciferase split into N-terminal and C-terminal fragments, wherein the probe is selected from any one or more of: (1) a probe comprising the KID domain of cyclic AMP response element-binding protein (CREB), the KIX domain of CREB-binding protein (CBP), the N-terminal fragment of luciferase (LucN), and the C-terminal fragment of luciferase (LucC) in one molecule; (2) (a) a probe consisting of two molecules, one of which comprises LucN and the KID domain and the other of which comprises LucC and the KIX domain, or (b) a probe consisting of two molecules, one of which comprises LucN and the KIX domain and the other of which comprises LucC and the KID domain; and (3) a probe consisting of two molecules, one of which comprises actin and LucN and the other of which comprises actin and LucC.

### TECHNICAL FIELD

The present invention relates to a probe for visualizing neural activity and to a transgenic animal having the probe therein.

### BACKGROUND ART

Although a wide variety of molecules are involved in brain neural activity, cAMP response element-binding protein (hereinafter, referred to as “CREB”) is known to be related to memory.

CREB is a transcriptional regulator and is activated through the phosphorylation of serine at residue 133.

The activated CREB binds to a CRE sequence (TGACGTCA) present in a gene promoter region and causes gene expression in the presence of a coupling factor CREB-binding protein (hereinafter, referred to as “CBP”).

Upon phosphorylation of CREB, the CREB forms a stable transcription complex with CBP through the hydrogen bond between the side chains of serine 113 of CREB KID (kinase inducible domain: phosphorylation site+CBP-binding site)) and tyrosine (Tyr) 658 of CBP KIX (CREB-binding site).

Meanwhile, actin is responsible for the control of cell shape or for cell motility through interaction with myosin. Its polymerization and depolymerization has been revealed to bidirectionally change the efficiency of synaptic transmission. Thus, the involvement of actin in neural activity including memory and learning has received attention.

On the other hand, a split luciferase method is known as a method for analyzing protein interaction (Patent Documents 1 and 2).
Patent Document 1: Japanese Patent Laid-Open No. 2007-325546
Patent Document 2: WO 02/008766

### DISCLOSURE OF THE INVENTION

### Problems to be Solved by the Invention

A split luciferase method described in Patent Documents 1 and 2 is a two-molecule type which comprises: dividing a firefly photoprotein luciferase into two domains, N-terminal and C-terminal fragments; fusing proteins A and B with these two fragments, respectively; and allowing two fusion proteins to be expressed in cells, wherein upon binding of the proteins A and B, the N- and C-termini of luciferase are in proximity to emit light again. This measurement method is free from a noise corresponding to autofluorescence in fluorescence observation and is suitable for measurement in live animals. However, this method cannot measure all protein-protein interactions only by simply preparing fusion proteins in accordance with the original method and requires detailed study on which region of the amino acid sequence of an individual protein is used and on how a luciferase protein is fused.

### Means for Solving the Problems

The present invention provides a probe capable of visualizing cyclic AMP response element-binding protein (CREB) activation or actin polymerization for the detailed study of protein-protein interaction involved in neural activity.

The probe refers to a probe consisting of two-molecule-type split luciferase capable of monitoring CREB activation, luciferase used in a one-molecule-type split luciferase method modified from the conventional two-molecule-type split luciferase method, or two-molecule-type split luciferase capable of visualizing actin polymerization. In this context, the one-molecule and two-molecule types mean the forms of one molecule and two molecules, respectively, at a protein level.

According to the method of the present invention, protein-protein interaction involved in neural activity can be visualized and observed. Specifically, the method of the present invention enables CREB activation at a single cell level or actin polymerization to be visualized and observed. Furthermore, this one-molecule system has facilitated the preparation of transgenic animals for observing protein-protein interaction in live animals.

A first aspect of the present invention relates to a probe for visualizing neural activity, the probe consisting of one or two molecule(s) and comprising luciferase split into N-terminal and C-terminal fragments. Specifically, the probe is selected from any one or more of the following (1) to (3):

(1) a probe comprising the KID domain of cyclic AMP response element-binding protein (CREB), the KIX domain of CREB-binding protein (CBP), the N-terminal fragment of luciferase (LucN), and the C-terminal fragment of luciferase (LucC) in one molecule;
(2) (a) a probe consisting of two molecules, one of which comprises LucN and the KID domain and the other of which comprises LucC and the KIX domain, or (b) a probe consisting of two molecules, one of which comprises LucN and the KIX domain and the other of which comprises LucC and the KID domain; and
(3) a probe consisting of two molecules, one of which comprises actin and LucN and the other of which comprises actin and LucN.

These probes may comprise a nuclear localization signal (NLS) and can comprise NLS, particularly in the N-terminal region.

The probe (1) is one-molecule-type split luciferase, wherein LucN, LucC, the KIX domain, and the KID domain can be linked in any order. For example, they can be linked in the following orders from the N-terminus:

### LucN-KID-KIX-LucC,

### LucC-KID-KIX-LucN,

### LucN-KIX-KID-LucC, and

### LucC-KIX-KID-LucC.

The probe can further comprise a linker sequence between LucN, LucC, the KIX domain, and the KID domain or on at least one of the N-terminal and the C-terminal sides of the probe molecule. For example, the linker sequence can be inserted between the KID domain and the KIX domain.

Examples of a modification of this probe include a probe which is one-molecule-type split luciferase free from the KIX domain. This probe comprises LucN-KID-LucC or LucC-KID-LucN, linked in this order from the N-terminus, and is capable of detecting the entire structural change of the KID domain.

The probe (2) is two-molecule-type split luciferase and is (a) a probe consisting of two molecules, one of which comprises LucN and the KID domain and the other of which comprises LucC and the KIX domain, or (b) a probe consisting of two molecules, one of which comprises LucN and the KIX domain and the other of which comprises LucC and the KID domain.

These probes are, for example, two-molecule-type split luciferase comprising LucC-KIX and LucN-KID respectively linked in this order from the N-terminus or two-molecule-type split luciferase comprising LucC-KID and LucN-KIX respectively linked in this order from the N-terminus.

The probe can further comprise a linker sequence between LucN, LucC, the KIX domain, and the KID domain or on the N-terminal and/or C-terminal sides of each probe molecule.

The probe (3) is two-molecule-type split luciferase and is a probe consisting of two molecules, one of which comprises actin and LucN and the other of which comprises actin and LucN. Examples of the probe (3) include:

two-molecule-type split luciferase comprising actin-LucN and actin-LucC,

two-molecule-type split luciferase comprising actin-LucN and LucC-actin,

two-molecule-type split luciferase comprising LucN-actin and LucC-actin, and

two-molecule-type split luciferase comprising LucN-actin and actin-LucC

(all the orders are viewed from the N-terminus).

The probe can further comprise a linker sequence between LucN, LucC, and actin or on the N-terminal and/or C-terminal sides of each probe molecule. For example, the linker can be contained between LucC and actin and/or between LucN and actin.

A second aspect of the present invention relates to a DNA encoding a probe of one or two protein molecule(s) for visualizing neural activity, the DNA comprising sequences respectively encoding luciferase split into N-terminal and C-terminal fragments. Specifically, the DNA is selected from any one of the following (1) to (3):

(1) a DNA comprising a sequence encoding the KID domain of cyclic AMP response element-binding protein (CREB), the KIX domain of CREB-binding protein (CBP), the N-terminal fragment of luciferase (LucN), and the C-terminal fragment of luciferase (LucC) as one molecule;
(2) (a) a DNA comprising a sequence encoding a molecule comprising LucN and the KID domain and a sequence encoding a molecule comprising LucC and the KIX domain, or
(b) a DNA comprising a sequence encoding a molecule comprising LucN and the KIX domain and a sequence encoding a molecule comprising LucC and the KID domain; and
(3) a DNA comprising a sequence encoding a molecule comprising actin and LucN and a sequence encoding a molecule comprising actin and LucN.

These DNAs may comprise a sequence encoding a nuclear localization signal (NLS). The DNAs can comprise a sequence encoding NLS, particularly in a region corresponding to the N-terminal region of the protein. Furthermore, these DNAs may comprise a marker gene such as a drug resistance gene for screening, a eukaryotic enhancer/promoter, and a poly-A addition signal sequence.

The DNA (1) is a DNA encoding the probe (1) of the first aspect; the DNA (2) is a DNA encoding the probe (2) of the first aspect; and the DNA (3) is a DNA encoding the probe (3) of the first aspect. The two sequences contained in the DNA encoding two-molecule-type split luciferase, such as the DNAs (2) and (3), may be carried by separate vectors, from which two molecules of the probe are respectively produced, or may be carried by one vector such that the DNA sequences respectively encoding two molecules of the probe flank an IRES sequence. Such a two-molecule probe-encoding DNA carried by one vector is preferable for preparing a transgenic animal described later.

A third aspect of the present invention relates to a visualization method comprising the steps of: producing the probe of the present invention in a nerve cell, the probe being one-molecule-type or two-molecule-type split luciferase; and measuring luminescence of the luciferase.

The probe can be produced, for example, in nerve cells in vivo and in vitro and can be expressed, for example, in the nerve cells of live transgenic animals.

Nerve cell excitation causes the conformational change of the probe of the present invention such that luciferase activity is restored to emit light. Since nerve cell excitation and luminescence are deemed to be in a proportional relationship, the number or site of excited nerve cells, the excited state, or the like can be measured quantitatively. According to this method, nerve cell excitation can be examined in vivo and in vitro, and nerve cell excitation in live animals can be observed based on the luminescence of luciferase because the toxicity of the luciferase is exceedingly low. For example, memory formation and neural activity can be visualized and studied in live animals.

In this visualization method, a rodent transfected with a DNA encoding the probe of the present invention, for example, a transgenic mouse prepared with a DNA encoding the probe of the present invention, can be used.

The use of such a rodent also allows screening of a substance promoting neural activity such as memory formation.

### Advantages of the Invention

Since CREB does not function as an intracellular dominant negative molecule by removing DNA-binding domains, dimerization domains, or the like from the polypeptide, luminescence associated with neural activity can be measured without impairing endogenous CBP activity. Moreover, the probe protein can be localized in the nucleus by fusing a nuclear localization domain to the N-terminus. Furthermore, the conversion of two-molecule-type split luciferase to one molecule achieves increased luminescence. Moreover, such a one-molecule probe is in a form suitable for preparing a transgenic animal. A more sensitively reacting transgenic animal can be prepared by phosphorylating the KID domain of the two-molecule-type CREB probe.

Moreover, the actin-linked two-molecule-type split luciferase of the present invention enables actin polymerization involved in neural activity such as memory formation to be directly observed in vivo in animals.

### BRIEF DESCRIPTION OF THE DRAWINGS

FIG. 1 is a graph showing results of Example 1. The graph depicts, as relative fluorescence, luminescence intensity obtained by administering 10 μM forskolin which increases intracellular cAMP after introduction of each fusion protein into HEK293 cells. The luminescence intensity of the fusion protein 1 was most increased;

FIG. 2 shows results of Example 2. The graph depicts change in luminescence intensity obtained by stimulating the fusion protein 1 expressed in nerve cells. The fusion protein 1 was stimulated at 0 minute, and the number of photons was directly measured. The potassium chloride (KCl) stimulation increased luminescence intensity by approximately 6 times. Specifically, the graph depicts the event in which the KCl stimulation significantly increased luminescence intensity within several tens of minutes;

FIG. 3 shows results of Example 3. The graph depicts the response of a KIX domain-free fusion protein (fusion protein 5) to KCl in nerve cells;

FIG. 4 is a graph showing results of Example 4. The graph depicts increase in luminescence after various stimulations to a split luciferase-CREB KID (phosphorylation domain) fusion protein expressed in nerve cells. A ratio between luminescence intensity from 0 minute (immediately after stimulation) to 1 minute and luminescence intensity from 40 minutes to 41 minutes was calculated and indicated as the ordinate of the graph. The black asterisks represent significant increase relative to a control, and the white asterisks represent significant increase relative to 50 mM KCl (based on at test);

FIG. 5 is a graph showing results of Example 5. Wild-type luciferase does not exhibit the response to KCl as shown in FIG. 4, demonstrating that the response of FIG. 4 occurs in a manner dependent on the inserted KID domain;

FIG. 6 is a graph showing results of Example 7. The graph depicts combinations of actin and split luciferase sequences and shows that a protein is most suitable in which the N-terminal or C-terminal fragment of split luciferase is fused on the N-terminal side of actin. In FIG. 6, FRB-FKBP split luciferase is a previously reported fusion protein;

FIG. 7 is a graph showing results of Example 8. The graph depicts the relationship between an actin polymerization inhibitor concentration and luminescence from split luciferase-actin fusion proteins bound via an IRES sequence and expressed in HEK293 cells;

FIG. 8 is a photograph showing results of Example 9. The photograph is an image of polymerized actin stained with rhodamine-phalloidin in the presence of varying concentrations of a polymerization inhibitor;

FIG. 9 is a graph showing results of Example 10. The graph depicts difference in luminescence intensity caused by exchanging sequences located before and after IRES;

FIG. 10 is a graph showing results of Example 7. The graph depicts results of observing luminescence intensity from 36 combinations of actin probes;

FIG. 11 is a graph showing results of Example 12. The graph depicts results of observing luminescence intensity from 72 combinations of two-molecule-type CREB probes; and

FIG. 12 is a graph showing results of Example 13. The graph depicts results of observing luminescence intensity from the combination of fusion proteins 31 and 45 in the presence or absence of forskolin.

### BEST MODE FOR CARRYING OUT THE INVENTION

### Luciferase

Luciferase derived from a freely selected organism can be used as the luciferase used in the present invention. Examples thereof include: insect luciferase such as firefly luciferase and Pyrophorus plagiophthalmus luciferase; Vargula hilgendorfii luciferase; Noctiluca scintillans luciferase; Metridia pacifica luciferase; Renilla luciferase; Watasenia scintillans luciferase; and variants thereof. The luciferase is preferably firefly-derived luciferase (EC1.13.12.7), more specifically Photinus pyralis-derived luciferase of SEQ ID NO: 1.

The luciferase used in the present invention is split into two domains, an N-terminal fragment (LucN) and a C-terminal fragment (LucC). For allowing the N-terminal and C-terminal fragments of the split luciferase to individually exhibit no fluorescence and to restore activity through the bond therebetween, the luciferase must be split such that its activity center is divided into two portions. Luciferase is known to be folded into two domains, a large N-terminal domain consisting of one β-barrel and two β-sheets and a C-terminal site, flanking a wide region including an activity center. Thus, the luciferase can be split at any flexible site of linkage between these two domains. This splitting is preferably performed in a nucleotide sequence encoding a protein of the luciferase gene. Examples thereof include splitting between bases 1245 and 1246.

### Actin

Examples of the actin used in the present invention include a protein encoded by mouse β-actin DNA (Accession No: BC138614).

### KID

Examples of the KID domain used in the present invention include DNA of bases 258 to 438 in a region encoding a protein of the mouse CREB gene (Accession No: BC021649) and a polypeptide encoded by the DNA.

### KIX

Examples of the KIX domain used in the present invention include DNA of bases 1755 to 1998 in a region encoding a protein of the mouse CBP gene (Accession No.: BC072594) and a polypeptide encoded by the DNA.

### Nuclear Localization Signal (NLS)

Examples of the nuclear localization signal used in the present invention include an SV40 nuclear localization signal. The amino acid sequence of the nuclear localization signal is as follows:

| | |
|---|---|
| LMDPKKKRKVDPKKKRKVG. | (SEQ ID NO: 2) |

### Internal Ribosomal Entry Site (IRES)

Examples of IRES used in the present invention include an IRES sequence (SEQ ID NO: 3) in a plasmid pIRES2-EGFP (Clontech Laboratories, Inc.).

### Linker

Examples of the linker used in the present invention include polypeptides having the following sequences:

| | |
|---|---|
| GGGGSGGGGSGGGGS, | (SEQ ID NO: 4) |
| and | |
| EAAAREAAARRAAAR. | (SEQ ID NO: 5) |

### Construction of Plasmid

Examples of plasmid construction methods include methods for incorporating a plurality of DNA fragments, for example, Multisite Gateway (registered trademark) System manufactured by Invitrogen Corp.

In The Multisite Gateway System, a DNA sequence encoding a portion of a fusion protein to be formed and a promoter region regulating gene expression are inserted in three plasmids (pDONR P4-P1R, pDONR221, pDONR P2R-P3 called donor vectors).

The Insertion Method is as Follows:

(1) Primers for PCR-amplifying an insert sequence are designed such that an attB sequence is added to both the ends of a PCR product.
(2) Two attP sequences located in each donor vector and the attB sequences of the PCR product react via an enzyme called BP Clonase (BP reaction) such that the PCR product is inserted between the attB sequences of the donor vector.
(3) These reactions proceed in vitro. Competent E. coli (TOP10; Invitrogen Corp.) is transformed with plasmids contained in this reaction solution and allowed to form colonies on an agar medium.
(4) Plasmids in one of these colonies are used in the next step.
(5) These donor vectors having the insert of the PCR product are called entry vectors. The three entry vectors are mixed with a destination vector (pDEST R4-R3) in vitro and reacted with LR Clonase such that three PCR products inserted in the entry vectors, respectively, are incorporated in series in the destination vector. By this procedure, the PCR products can be incorporated in the order of pDONR P4-P1R, pDONR221, and pDONR P2R-P3 to accurately obtain plasmids expressing the fusion protein of interest.
(6) The plasmids thus obtained finally contain an ampicillin resistance gene, an SV40 eukaryotic enhancer/promoter, and a poly-A addition signal sequence.

QIA prep spin miniprep kit manufactured by Qiagen can be used in plasmid purification.

When plasmids are constructed using Multisite Gateway System manufactured by Invitrogen Corp., a particular amino acid sequence may be added to the fusion protein. For example, for actin, an amino acid sequence KGGRADPAFLYKVE (SEQ ID NO: 58) is added between the sequence of actin and the N-terminal or C-terminal fragment of luciferase. This addition of the particular amino acid sequence does not influence the effect of the present invention.

### Construction of Plasmid 2

In the present invention, site-directed mutagenesis can also be utilized in plasmid construction. Specifically, for example, KOD plus mutagenesis kit manufactured by TOYOBO CO., LTD. may be used.

### Donor Vectors

(a) pDONR P4-P1R

A promoter sequence or a promoter sequence linked to a nuclear localization signal sequence can be incorporated in this vector for use. An SV40 enhancer or promoter encoded by a plasmid pGL4.13 manufactured by Promega Corp. can be used as the promoter sequence.

(b) pDONR221 Donor Vector

The DNA sequence inserted therein is, for example, a sequence encoding the following:

actin,
luciferase (wild-type),

### LucN,

### LucC,

KID sequence,
KIX sequence,
LucN-KID sequence-linker sequence,
LucN-KIX sequence-linker sequence,
LucC-KID sequence-linker sequence, or
LucN-KID sequence.

When a plurality of sequences are inserted in pDONR221, these sequences can be consecutively inserted in advance in a plasmid pLITMUS28 (New England Biolabs, Inc.) using restriction sites in its multicloning site. Then, these consecutive sequences can be amplified by PCR and inserted in pDONR221 through BP reaction.

The sequences except for that encoding luciferase (wild-type) are free from a termination codon.

(c) pDONR P2R-P3 Donor Vector

The DNA sequence inserted therein can be a sequence encoding the following:

actin,

### LucN,

### LucC,

KID sequence, or
KIX sequence.

All the sequences contain a termination codon.

When the intended sequence is inserted to the pDONR P4-P1R plasmid through BP reaction, a primer set can be used which is obtained by adding attB4-forward sequence: 5′-GGGGACAACTTTGTATAGAAAAGTTGAA-3′ (SEQ ID NO: 59)

to the 5′ end of a forward primer corresponding to the intended DNA sequence, and adding
attB1-reverse sequence: 5′-GGGGACTGCTTTTTTGTACAAACTTGA-3′ (SEQ ID NO: 60)
to the 3′ end of a reverse primer corresponding to the intended DNA sequence.

When the intended sequence is inserted to the pDONR221 plasmid through BP reaction, a primer set can be used which is obtained by adding attB1-forward sequence: 5′-GGGGACAAGTTTGTACAAAAAAGCAGGCTTT-3′ (SEQ ID NO: 61)

to the 5′ end of a forward primer corresponding to the intended DNA sequence, and adding
attB2-reverse sequence: 5′-GGGGACCACTTTGTACAAGAAAGCTGGGTT-3′ (SEQ ID NO: 62)
to the 3′ end of a reverse primer corresponding to the intended DNA sequence.

When the intended sequence is inserted to the pDONR P2R-P3 plasmid through BP reaction, a primer set can be used which is obtained by adding attB2-forward sequence: 5′-GGGGACAGCTTTCTTGTACAAAGTGGAA-3′ (SEQ ID NO: 63)

to the 5′ end of a forward primer corresponding to the intended DNA sequence, and adding
attB3-reverse sequence: 5′-GGGGACAACTTTGTATAATAAAGTTGT-3′ (SEQ ID NO: 64)
to the 3′ end of a reverse primer corresponding to the intended DNA sequence.

pENTR/D-TOPO (Invitrogen Corp.) can be used, instead of pDONR221, as a plasmid for donor vector preparation in Multisite Gateway. As in pDONR221, pENTR/D-TOPO (Invitrogen Corp.) is a plasmid for preparing the donor vector in Multisite Gateway System but is different from pDONR221 in a gene insertion method. For pDONR221, a PCR product is incorporated to the plasmid using BP reaction. By contrast, for pENTR/D-TOPO, a PCR product is incorporated to the plasmid using DNA binding catalyzed by topoisomerase. Thus, the incorporation of a PCR product to pDONR221 requires adding the attB sequence to the ends of both primers, whereas a blunt-ended PCR product can be incorporated directly to pENTR/D-TOPO.

### Confirmation of Luminescent Function

To confirm the luminescence ability of the probe of the present invention, for example, for CREB, HEK293 cells (human kidney-derived cell line) are cultured. After 2 days into culture, the plasmid for the fusion protein is gene-transferred to the cells. After 3 days into culture, forskolin, which phosphorylates CREB, is added to the medium, and the cultured cells are separated on the next day. The separated cells are transferred to a plate. After addition of luciferin, the luminescence intensity can be measured using a luminometer.

### Transgenic Mouse

A transgenic mouse can be prepared according to the following procedures:

(1) linear DNA is prepared, in which three components, i.e., a promoter for inducing expression, a gene to be expressed, and a poly-A signal for mRNA polyadenylation, are linked in series;
(2) the prepared linear DNA is microinjected to artificially fertilized eggs, which are then transplanted into the womb of another pseudopregnant mother; and
(3) Of the fertilized eggs, those having the injected DNA incorporated in the genomic DNA are born as a transgenic mouse.

### EXAMPLES

Hereinafter, the present invention will be described more specifically with reference to Examples. However, the present invention is not limited to these Examples by any means.

### Reference Example 1

### Luciferase

For the luciferase used in the present invention, the coding region of the firefly luciferase gene of a plasmid pGL4.13 (Promega Corp.) was amplified by PCR using the following primers:

(SEQ ID NO: 6)

| | |
|---|---|
| Forward primer | 5′-ATGGAAGATGCCAAAAACATTAAGA-3′, |
| and | |

(SEQ ID NO: 7)

| | |
|---|---|
| Reverse primer | 5′-TTACACGGCGATCTTGCCGCCCTTC-3′. |

### Reference Example 2

### Split Luciferase: LucN

For LucN, a sequence of bases 1 to 1245 in the firefly luciferase sequence was obtained by amplification using a forward primer having a sequence of bases 1 to 25 (5′-ATGGAAGATGCCAAAAACATTAAGA-3′ (SEQ ID NO: 6)) thereof and a reverse primer having a complementary sequence (5′-GTCCTTGTCGATGAGAGCGTTTGTA-3′ (SEQ ID NO: 9)) of a sequence of bases 1221 to 1245 (5′-TACAAACGCTCTCATCGACAAGGAC-3′ (SEQ ID NO: 8)) thereof, as a PCR primer set corresponding to the DNA sequence. In this PCR, a plasmid pGL4.13 was used as a template sequence.

### Reference Example 3

### Split Luciferase: LucC

For LucC, a sequence of bases 1246 to 1653 in the firefly luciferase sequence was obtained by amplification using a sequence of bases 1246 to 1270 (5′-GGCTGGCTGCACAGCGGCGACATCG-3′ (SEQ ID NO: 10)) thereof and a complementary sequence (5′-TTACACGGCGATCTTGCCGCCCTTC-3′ (SEQ ID NO: 7)) of a sequence of bases 1629 to 1653 (5′-GAAGGGCGGCAAGATCGCCGTGTAA-3′ (SEQ ID NO: 11)) thereof, as a PCR primer set corresponding to the DNA sequence. In this PCR, a plasmid pGL4.13 was used as a template sequence.

### Reference Example 4

### Actin

A sequence of bases 1 to 1128 in an actin sequence was obtained by amplification using a forward primer having a sequence of bases 1 to 25 (5′-ATGGATGACGATATCGCTGCGCTGG-3′ (SEQ ID NO: 12)) thereof and a reverse primer having a complementary sequence (5′-CTAGAAGCACTTGCGGTGCACGATG-3′ (SEQ ID NO: 14)) of a sequence of bases 1104 to 1128 (5′-CATCGTGCACCGCAAGTGCTTCTAG-3′ (SEQ ID NO: 13)) thereof, as a PCR primer set corresponding to the DNA sequence. In this PCR, cDNA obtained by purifying total RNA from adult mouse (C57BL6) cerebral cortex using RNeasy mini Kit (Qiagen) and performing the reverse transcription reaction of the total RNA using SuperScript III kit (Invitrogen Corp.) was used as a template sequence.

### Reference Example 5

### KID Sequence

A sequence of bases 258 to 438 in a CREB protein-encoding sequence was obtained by amplification using a forward primer having a sequence of bases 258 to 282 (5′-CAGATTTCAACTATTGCAGAAAGTG-3′ (SEQ ID NO: 15)) thereof and a reverse primer having a complementary sequence (5′-AGTCTCCTCTTCTGACTTTTCTTCT-3′ (SEQ ID NO: 17)) of a sequence of bases 414 to 438 (5′-AGAAGAAAAGTCAGAAGAGGAGACT-3′ (SEQ ID NO: 16)) thereof, as a PCR primer set corresponding to the DNA sequence. In this PCR, cDNA obtained by purifying total RNA from adult mouse (C57BL6) cerebral cortex using RNeasy mini Kit (Qiagen) and performing the reverse transcription reaction of the total RNA using SuperScript III kit (Invitrogen Corp.) was used as a template sequence.

### Reference Example 6

### KIX Sequence

A sequence of bases 1755 to 1998 in a CBP protein-encoding sequence was obtained by amplification using a forward primer having a sequence of bases 1755 to 1779 (5′-GGTGTTCGAAAAGGCTGGCATGAAC-3′ (SEQ ID NO: 18)) thereof and a reverse primer having a complementary sequence (5′-TTCTTCTAGTTCTTTTTGTATTTTA-3′ (SEQ ID NO: 20)) of a sequence of bases 1974 to 1998 (5′-TAAAATACAAAAAGAACTAGAAGAA-3′ (SEQ ID NO: 19)) thereof, as a PCR primer set corresponding to the DNA sequence. In this PCR, cDNA obtained by purifying total RNA from adult mouse (C57BL6) cerebral cortex using RNeasy mini Kit (Qiagen) and performing the reverse transcription reaction of the total RNA using SuperScript III kit (Invitrogen Corp.) was used as a template sequence.

### Reference Example 7

### NLS

The nucleotide sequence of an nuclear localization signal (NLS) is as follows:

(SEQ ID NO: 21)
CTTATGGATCCAAAAAAGAAGAGAAAGGTAGACCCTAAGAAAAAGAGG
AAAGTTGGG.

This sequence and its complementary sequence were mixed in one test tube and hybridized by heating to 95° C. and then gradually cooled to 37° C. over 1 hour. The double-stranded DNA thus hybridized was inserted and cloned in plasmids using Zero blunt TOPO kit (Invitrogen Corp.), which is a kit for cloning blunt-ended double-stranded DNA.

The DNA sequence of the nuclear localization signal was further inserted to a plasmid pDONR P4-P1R having an insert of an SV40 enhancer or promoter. A HindIII restriction site located at base 416 of the SV40 enhancer or promoter was used to perform PCR amplification using a forward primer having a sequence of bases 1 to 25 (5′-CTTATGGATCCAAAAAAGAAGAGAA-3′ (SEQ ID NO: 22)) of the nuclear localization signal sequence, plus a HindIII site added to the 5′ end of this sequence portion corresponding to the DNA, and a reverse primer having a complementary sequence (5′-CCCAACTTTCCTCTTTTTCTTAGGG-3′ (SEQ ID NO: 24)) of a sequence of bases 33 to 57 (5′-CCCTAAGAAAAAGAGGAAAGTTGGG-3′ (SEQ ID NO: 23)) thereof, plus a HindIII site added to the 5′-end of this sequence portion corresponding to the DNA. In this PCR, a plasmid prepared using Zero blunt TOPO kit was used as template DNA. The amplified sequence was inserted in the HindIII site.

### Reference Example 8

### IRES Sequence

A sequence (SEQ ID NO: 3) in “pIRES2-EGFP Vector” manufactured by Clontech Laboratories, Inc. was used as an IRES sequence.

For PCR amplification, a forward primer 5′-GATCCGCCCCTCTCCCTCCCCC-3′ (SEQ ID NO: 25) and a reverse primer 5′-GGTTGTGGCCATATTATCATCGTG-3′ (SEQ ID NO: 26) were used as primer sites corresponding to the DNA sequence. For PCR intended for insertion in plasmids, restriction sites (EcoRI and BamHI were used this time) were added to the 5′ ends of these primer sequences, respectively. The amplified sequence was inserted in the restriction sites of plasmids.

### Reference Example 9

### Linker Sequence

The nucleotide sequence of a linker is as follows:

(SEQ ID NO: 27)
GGAGGTGGGGGTAGTGGGGGCGGAGGTAGCGGTGGCGGTGGTAGT.

This sequence and its complementary sequence were mixed in one test tube and hybridized by heating to 95° C. and then gradually cooled to 37° C. over 1 hour. The double-stranded DNA thus hybridized was inserted and cloned in plasmids using Zero blunt TOPO kit (Invitrogen Corp.), which is a kit for cloning blunt-ended double-stranded DNA.

For PCR amplification,

a forward primer 5′-GGAGGTGGGGGTAGTGGGGGC-3′ (SEQ ID NO: 28), and a reverse primer 5′-ACTACCACCGCCACCGCTACC-3′ (SEQ ID NO: 29)
were used as primer sites corresponding to the DNA sequence. For PCR intended for insertion in plasmids, restriction sites were added to the 5′ ends of these primer sequences, respectively. The amplified sequence was inserted in the restriction sites of plasmids.

### Reference Example 10

### SV40 Enhancer/Promoter

The SV40 enhancer/promoter site of a plasmid pGL4.13 (Promega Corp.) was amplified by PCR for use.

A sequence of bases 1 to 419 in the SV40 enhancer or promoter sequence was obtained by amplification using a forward primer having a sequence of bases 1 to 25 (5′-GCGCAGCACCATGGCCTGAAATAAC-3′ (SEQ ID NO: 30)) thereof and a reverse primer having a complementary sequence (5′-AAGCTTTTTGCAAAAGCCTAGGCCT-3′ (SEQ ID NO: 32)) of a sequence of bases 395 to 419 (5′-AGGCCTAGGCTTTTGCAAAAAGCTT-3′ (SEQ ID NO: 31)) thereof, as a PCR primer set corresponding to the DNA sequence. In this PCR, a plasmid pGL4.13 was used as a template sequence. For inserting this sequence in pDONR P4-P1R, attB4-forward or attB1-reverse sequence-tagged primers corresponding to the DNA sequence were used in PCR.

For plasmid construction,

pDONR P4-P1R having an insert of SV40 promoter-NLS,
pDONR221 having an insert of LucN-KID-linker-KIX, and
pDONR P2R-P3 having an insert of LucC
were used to prepare final plasmids using Multisite Gateway.

However, prior to insertion of LucN-KID-linker-KIX to pDONR221 through BP reaction, these sequences were consecutively inserted into pLITMUS28 (New England Biolabs, Inc.) using restriction sites in its multicloning site. The restriction sites were as follows:

(SpeI)-LucN-(EcoRI)-KID-(NcoI)-Linker-(AgeI)-KIX-
(SacI).

The restriction enzymes are shown within the parentheses. Each insert was amplified by PCR. For this PCR, the restriction site sequences were respectively added to the 5′ ends of primers for each sequence. The amplified PCR fragment and pLITMUS28 were separately cleaved with restriction enzymes, and the cleaved fragment was inserted to the plasmid using ligase.

The sequence LucN-KID-linker-KIX was completed in pLITMUS28 and then amplified again by PCR. This PCR was performed using primers 5′-terminally tagged with an attB sequence for insertion in pDONR221. Then, the amplified sequence was inserted into pDONR221 through BP reaction.

### Example 1

HEK cells were transfected with DNA sequences encoding proteins comprising the phosphorylation domain KID of CREB protein, the KIX domain of CBP protein known to bind to KID, and split luciferase fused in combinations shown below. Increase in luminescence intensity obtained by administering forskolin was observed.

Fusion protein 1:
NLS-LucN-KID-GGGGSGGGGSGGGGS-KIX-LucC
Fusion protein 2:
NLS-LucN-KID-EAAAREAAAREAAAR-KIX-LucC
Fusion protein 3:
NLS-LucN-KIX-EAAAREAAAREAAAR-KID-LucC
Fusion protein 4:
NLS-LucC-KID-GGGGSGGGGSGGGGS-KIX-LucN

The luminescence measurement was performed as follows:

HEK293 was cultured in a plastic dish (Falcon 12-well dish). The medium used was 1 mL/well of a Dulbecco's modified eagle's medium (DMEM) supplemented with 10% bovine serum. The culture was performed in an incubator under conditions involving 37° C., 5% CO₂, and 100% humidity.

Plasmids respectively encoding the fusion proteins 1 to 4 were prepared. On two days into culture, these plasmids were gene-transferred into the HEK 293 cells. The gene transfer was performed using Lipofectamine 2000 (Invitrogen Corp.). An Opti-MEM medium and each plasmid DNA were mixed at a ratio of 125 μL:1 μg and incubated at room temperature for 5 minutes. Aside from this, an Opti-MEM medium and Lipofectamine 2000 were mixed at a ratio of 125 μL:2 μL and incubated at room temperature for 5 minutes in the same way as above. Both of the mixtures were mixed and incubated at room temperature for 20 minutes to form a DNA-Lipofectamine 2000 complex. The mixed solution was added dropwise at a concentration of 250 μl/well to the medium and subsequently cultured.

After 3 days in culture, forskolin was added at a final concentration of 10 μM to the medium, followed by additional one day of culture. After discarding of the medium, 50 μL of PBS was added thereto, and the cultured cells were scraped from the dish using a cell scraper made of rubber and transferred to a 96-well plate. Furthermore, 50 μL of luciferin (Bright-Glo; Promega Corp.) was added thereto, and the luminescence intensity of each well was measured using a luminometer (TECAN Group Ltd.).

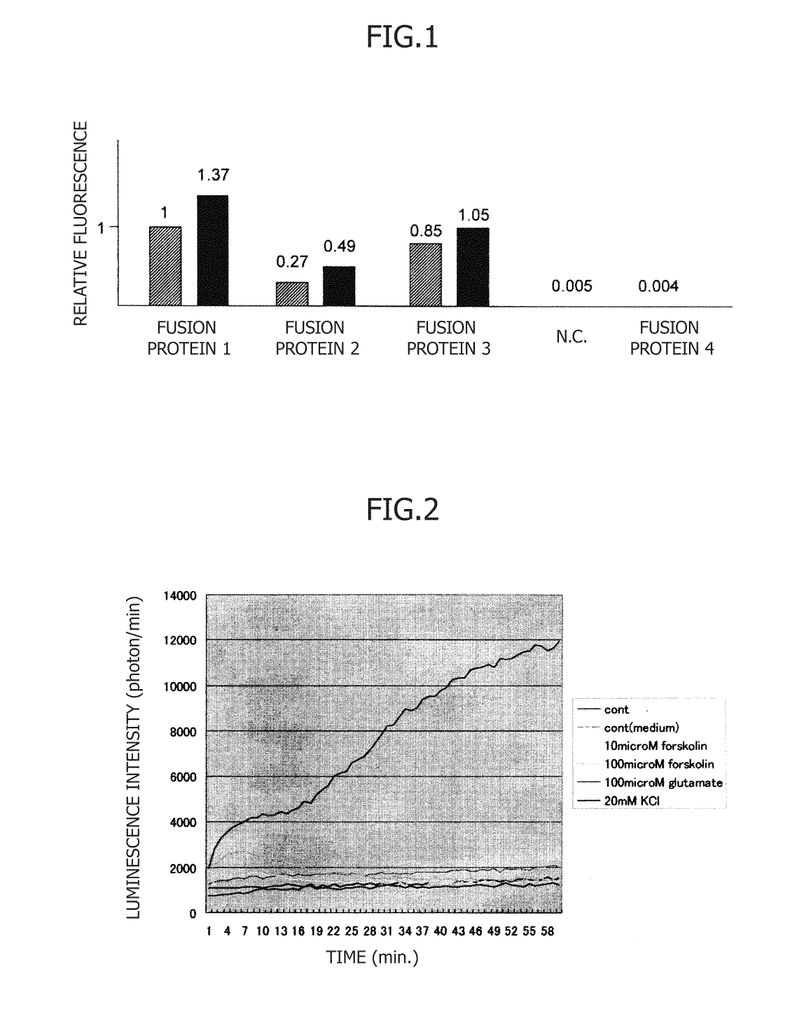

The results are shown in FIG. 1. For each of the fusion proteins 1 to 4 and a negative control, the left bar (indicated in gray) in the graph represents the results obtained without the addition of forskolin, and the right bar (indicated in black) represents the results obtained with the addition of forskolin at a final concentration of 10 μM. As a result, the fusion protein 1 exhibited the strongest luminescence intensity.

### Example 2

On day 18 of pregnancy, a rat fetus was taken out of a pregnant rat, and the brain was separated therefrom in cold PBS. Furthermore, brain slices containing hippocampal nerve cells were separated from the cerebrum. The separated hippocampal cells were reacted with 0.125% trypsin (protease) at room temperature for 20 minutes in a test tube such that adhesion factors on the cell surface were degraded to attenuate cell-cell adhesion. Then, the test tube was left standing for trypsin removal. After precipitation of the brain slices in the bottom of the test tube, the trypsin solution as a supernatant was removed by aspiration. Subsequently, a DMEM medium containing 10% serum was added to the test tube. The test tube was left standing again, and the supernatant was removed. The brain slices were dissociated into individual cells by repeating approximately 10 times aspiration and dropping using a plastic dropper. Then, the cells were cultured in a plastic dish. The conditions of the medium were the same as in Example 1.

On culture day 4, the plasmid encoding the fusion protein 1 of Example 1 was gene-transferred into the cells. The gene transfer method was performed using Lipofectamine 2000 in the same way as in the HEK293 cells. Two days after the gene transfer, the medium was replaced by an Opti-MEM medium (Invitrogen Corp.) containing 0.5 mM luciferin EF (Promega Corp.), a luminescent substrate of luciferase. The luminescence intensity was measured using a luminometer AEQUORIA (Hamamatsu Photonics K.K.). This apparatus counts the number of photons generated from cells during culture placed together with a plastic dish in a dark box. The measurement was performed for 1 consecutive hour, and the luminescence intensity at 1-minute intervals was plotted on the ordinate of a graph. Immediately before the measurement, the cells were stimulated with forskolin, glutamate, KCl, or the like.

Forskolin works to increase the amount of intracellular cAMP. Glutamate is a main neurotransmitter of hippocampal nerve cells and excites the nerve cells. Upon addition of KCl to an extracellular fluid, the ion balance between the cells and their surroundings was changed to depolarize the nerve cells. The nerve cell can thereby be excited due to calcium entry into the cells or the like.

The results are shown in FIG. 2. The luminescence intensity was particularly increased by the addition of KCl to the extracellular fluid and increased by approximately 6 times compared to that before addition.

### Example 3

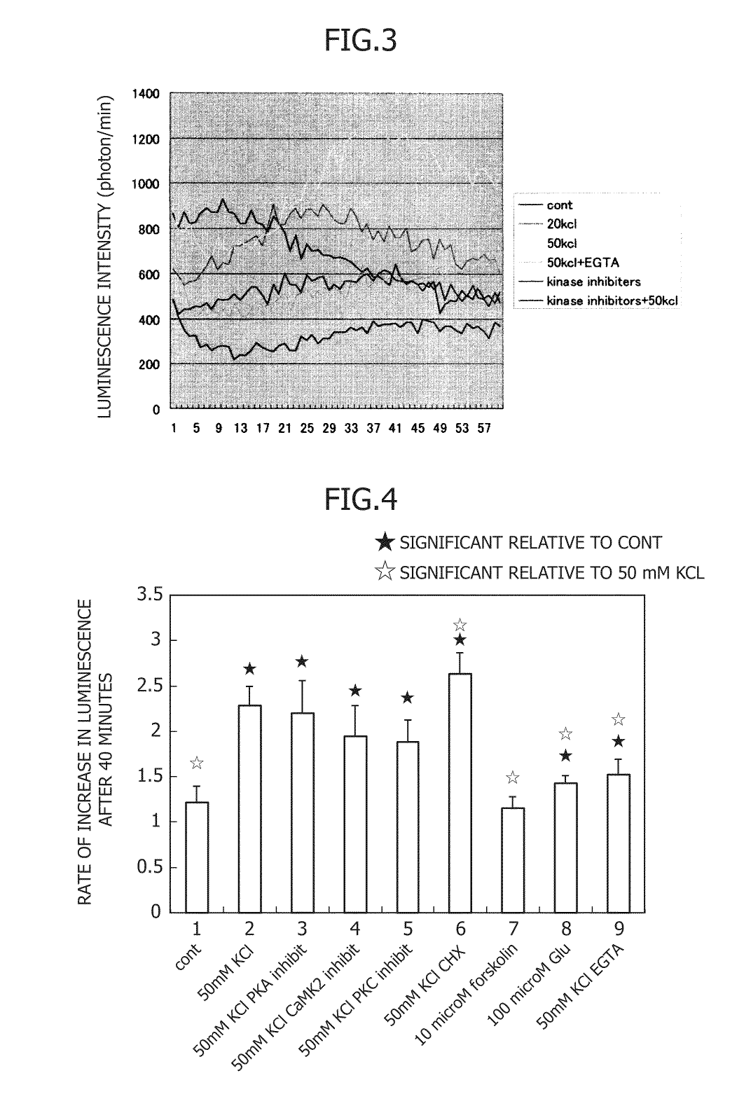

In the same experimental system as in Example 2, from a plasmid encoding a fusion protein 1 of Example 1, a fusion protein 5 (NLS-LucN-KID-LucC), free from the linker and the KIX domain was expressed in nerve cells and measured for its response to various stimulations (FIG. 3).

The fusion protein free from the KIX domain also exhibits the same response to KCl in nerve cells. However, a fusion protein free from the KID domain exhibits no luminescence (data not shown). This demonstrated that the KCl stimulation causes the structural change of the KID domain to increase luciferase activity. It is the consensus view that stimulation to nerve cells would cause the structural change of the KID domain.

### Example 4

In the same experimental system as in Example 2, the response of nerve cells with the expressed fusion protein 5 to various stimulations (50 mM KCl, 50 mM KCl and PKA inhibitor, 50 mM KCl and CaMK₂ inhibitor, 50 mM KCl and PKC inhibitor, 50 mM KCl and CHX, 10 μM forskolin, 100 μM glutamate, and 50 mM KCl and EGTA) was confirmed. The results are shown in FIG. 4. The KCl simulation of the nerve cells increases the luminescence intensity. Moreover, EDTA suppresses increase in luminescence to some extent, demonstrating that the occurrence of calcium entry into the nerve cells after KCL stimulation is important.

### Example 5

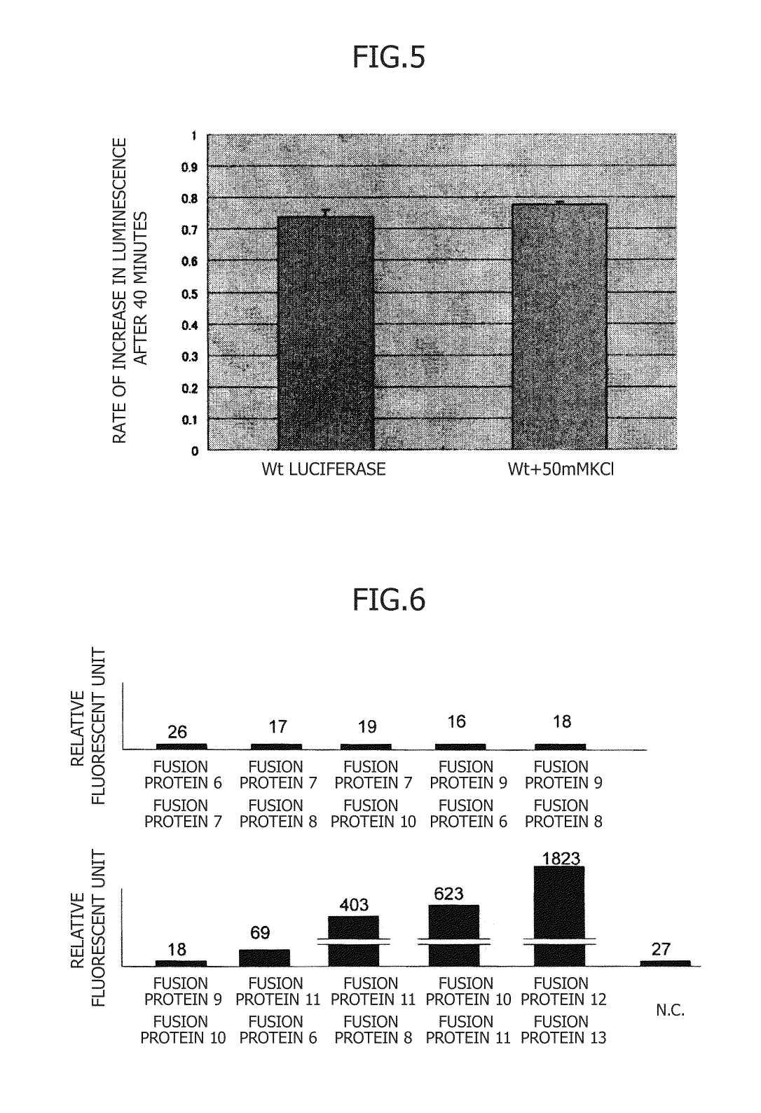

This Example was intended for a control experiment using wild-type firefly luciferase. In the same experimental system as in Example 2, the plasmid for wild-type firefly luciferase (Promega Corp.) was gene-transferred to nerve cells, which were then stimulated with KCl (FIG. 5).

The wild-type luciferase does not exhibit the response to KCl as shown in FIG. 4, demonstrating that the response of FIG. 4 occurs in a manner dependent on the inserted KID domain.

### Example 6

(1) Plasmids were constructed using a Multisite Gateway System to obtain fusion proteins having the actin sequence (Actin) and the C-terminal fragment of luciferase (LucC) or the N-terminal fragment of luciferase (LucN) shown below. In the description below, “KGGRADPAFLYKVE” (SEQ ID NO: 58) is an amino acid sequence added by the Multisite Gateway System. Moreover, FKBP means an FK506-binding protein, and FRB means FKBP-rapamycin-binding domain (mTOR (mammalian target of rapamycin)).

Fusion protein 6:
Actin-KGGRADPAFLYKVE-LucC (Actin-LucC)
Fusion protein 7:
Actin-KGGRADPAFLYKVE-LucN (Actin-LucN)
Fusion protein 10:
LucC-KGGRADPAFLYKVE-Actin (LucC-Actin)
Fusion protein 11:
LucN-KGGRADPAFLYKVE-Actin (LucN-Actin)
Fusion protein 12:
FRB-LucN
Fusion protein 13:
FKBP-LucC

(2) A fusion protein 9 was prepared with the DNA sequence of the fusion protein 7 as a template using KOD plus mutagenesis kit. Specifically, the plasmid for the fusion protein 7 was used as a template to perform PCR using a primer comprising the reverse primer binding to the tail of the N-terminal fragment of luciferase, plus a sequence encoding the first half of the linker sequence (added to the 5′ end of the reverse primer) 5′-CGCCCCCACTACCCCCACCTCCGTCCTTGTCGATGAGAGCGTTTGTA-3′ (SEQ ID NO: 33), and

a primer comprising the forward primer recognizing the head of the actin sequence, plus a sequence encoding the last half of the linker sequence 5′-GAGGTAGCGGTGGCGGTGGTAGTATGGATGACGATATCGCTGCGCTGG-3′ (SEQ ID NO: 34).
Subsequently, the fusion protein plasmid of the template was degraded with an enzyme DpnI selectively digesting only methylated DNA. Furthermore, the PCR product was ligated at both the ends and cloned in E. coli to obtain the following fusion protein:

fusion protein 9: Actin-GGGGSGGGGSGGGGS-LucN.

(3) A fusion protein 8 was prepared with the DNA sequence of the fusion protein 2 as a template using KOD plus mutagenesis kit. Specifically, the plasmid for the fusion protein 7 was used as a template to perform PCR using a primer comprising the reverse primer binding to the tail of the C-terminal fragment of luciferase, plus a sequence encoding the first half of the linker sequence (added to the 5′ end of the reverse primer) 5′-CGCCCCCACTACCCCCACCTCCGAAGGGCGGCAAGATCGCCGTG-3′ (SEQ ID NO: 35), and

a primer comprising the forward primer recognizing the head of the actin sequence, plus a sequence encoding the last half of the linker sequence 5′-GAGGTAGCGGTGGCGGTGGTAGTATGGATGACGATATCGCTGCGCTGG-3′ (SEQ ID NO: 34).
Subsequently, the fusion protein plasmid of the template was degraded with an enzyme DpnI selectively digesting only methylated DNA. Furthermore, the PCR product was ligated at both the ends and cloned in E. coli to obtain the following fusion protein:

fusion protein 8: Actin-GGGGSGGGGSGGGGS-LucC.

(4) A fusion protein 16 was prepared with the DNA sequence of the fusion protein 11 as a template. Specifically, the plasmid for the fusion protein 16 was used as a template to perform PCR using a reverse primer comprising the tail of the actin sequence linked to a sequence encoding the first half of a linker (GGGGSGGGGS) 5′-ACTACCCCCACCTCCGAAGCACTTGCGGTGCACGATG-3′ (SEQ ID NO: 36), and

a forward primer comprising a KGGRADPA moiety (resulting from the Multisite Gateway of the fusion protein 5)-encoding sequence linked to a sequence encoding the last half of the linker (GGGGSGGGGS) 5′-GGTGGCGGTGGTAGTAAGGGTGGGCGCGCCGAGCCAGCT-3′ (SEQ ID NO: 37).
Subsequently, the fusion protein plasmid of the template was degraded with an enzyme DpnI selectively digesting only methylated DNA. Furthermore, the PCR product was ligated at both the ends and cloned in E. coli to obtain the following fusion protein having a linker sequence GGGGSGGGGSKGGRADPAFLYKVE (SEQ ID NO: 65):

fusion protein 16:
Actin-GGGGSGGGGSKGGRADPAFLYKVE-LucN.

(5) A fusion protein 17 was prepared with the DNA sequence of the fusion protein 10 as a template using the same primer set as in a fusion protein 14:

fusion protein 17:
Actin-GGGGSGGGGSKGGRADPAFLYKVE-LucC.

(6) A fusion protein 18 was prepared with the DNA sequence of the fusion protein 11 as a template. Specifically, the plasmid for the fusion protein 18 was used as a template to perform PCR using a reverse primer comprising the sequence of the tail of the actin sequence 5′-GAAGCACTTGCGGTGCACGATG-3′ (SEQ ID NO: 38), and a forward primer comprising the head of the N-terminal fragment of luciferase 5′-ATGGAAGATGCCAAAAACATTAAGA-3′ (SEQ ID NO: 39).

Subsequently, the fusion protein plasmid of the template was degraded with an enzyme DpnI selectively digesting only methylated DNA. Furthermore, the PCR product was ligated at both the ends and cloned in E. coli to obtain the following fusion protein:

fusion protein 18: Actin-LucN.

(7) A fusion protein 19 was prepared with the DNA sequence of the fusion protein 10 as a template. Specifically, the plasmid for the fusion protein 19 was used as a template to perform PCR using a reverse primer comprising the sequence of the tail of the actin sequence 5′-GAAGCACTTGCGGTGCACGATG-3′ (SEQ ID NO: 38), and a forward primer comprising the head of the C-terminal fragment of luciferase 5′-GGCTGGCTGCACAGCGGCGACATCG-3′ (SEQ ID NO: 40).

Subsequently, the fusion protein plasmid of the template was degraded with an enzyme DpnI selectively digesting only methylated DNA. Furthermore, the PCR product was ligated at both the ends and cloned in E. coli to obtain the following fusion protein:

fusion protein 19: Actin-LucC.

(8) A fusion protein 20 was prepared with the DNA sequence of the fusion protein 11 as a template. Specifically, the plasmid for the fusion protein 20 was used as a template to perform PCR using a reverse primer comprising the sequence of the tail of the actin sequence linked to a sequence encoding the first half of the linker sequence 5′-CCGCCCCCACTACCCCCACCTCCGAAGCACTTGCGGTGCACGATG-3′ (SEQ ID NO: 41), and

a forward primer comprising the head of the N-terminal fragment of luciferase linked to a sequence encoding the last half of the linker sequence 5′-AGGTAGCGGTGGCGGTGGTAGTGGCTGGCTGCACAGCGGCGACATCG-3′ (SEQ ID NO: 42).
Subsequently, the fusion protein plasmid of the template was degraded with an enzyme DpnI selectively digesting only methylated DNA. Furthermore, the PCR product was ligated at both the ends and cloned in E. coli to obtain the following fusion protein:

fusion protein 20: Actin-GGGGSGGGGSGGGGS-LucN.

(9) A fusion protein 21 was prepared with the DNA sequence of the fusion protein 10 as a template. Specifically, the plasmid for the fusion protein 21 was used as a template to perform PCR using a reverse primer comprising the sequence of the tail of the actin sequence linked to a sequence encoding the first half of the linker sequence 5′-CCGCCCCCACTACCCCCACCTCCGAAGCACTTGCGGTGCACGATG-3′ (SEQ ID NO: 41), and

a forward primer comprising the head of the C-terminal fragment of luciferase linked to a sequence encoding the last half of the linker sequence 5′-AGGTAGCGGTGGCGGTGGTAGTGGCTGGCTGCACAGCGGCGACATCG-3′ (SEQ ID NO: 42).
Subsequently, the fusion protein plasmid of the template was degraded with an enzyme DpnI selectively digesting only methylated DNA. Furthermore, the PCR product was ligated at both the ends and cloned in E. coli to obtain the following fusion protein:

fusion protein 21: Actin-GGGGSGGGGSGGGGS-LucC.

### Example 7

(1) Combinations of the plasmids for the following fusion proteins consisting of actin and split luciferase were gene-transferred to HEK293T cells:

fusion protein 6: Actin-LucC,
fusion protein 7: Actin-LucN,
fusion protein 8: Actin-Linker-LucC,
fusion protein 9: Actin-Linker-LucN,
fusion protein 10: LucC-Actin,
fusion protein 11: LucN-Actin,
fusion protein 12: FRB-LucN,
and
fusion protein 13: FKBP-LucC.

The combination of the fusion proteins 12 and 13 serves as a positive control.

In the same way as in Example 1, the cells were scraped two days after the gene transfer, and the luminescence was measured using a luminometer. As a result, it was demonstrated that the combination of the proteins comprising the N-terminal or C-terminal fragment of the split luciferase fused on the N-terminal side of actin (fusion proteins 10 and 11) is most suitable (FIG. 6).

(2) Two (containing the N-terminal and C-terminal fragments of luciferase, respectively) selected from the plasmids for the fusion proteins 6 to 11 and 16 to 21 were gene-transferred to HEK293 cells, and the luminescence intensity (the number of photons observed per 10 minutes) was observed. The results are shown in FIG. 10.

### Example 8

For preparing a form suitable for transgenic mouse preparation, plasmids were constructed for a fusion protein comprising two fusion proteins bound via an IRES sequence (the resultant fusion protein is referred to as a fusion protein 13: LucN-Actin-IRES-LucC-actin). LucC-Actin and LucN-Actin are thereby translated from one mRNA and expressed in cells. The gene transfer was performed in the same way as in Example 1. Moreover, in this experiment, an actin polymerization inhibitor latrunculin A was added into a medium 3 hours before luminescence measurement. Moreover, the luminescence measurement used a luminometer AEQUORIA (Hamamatsu Photonics K.K.).

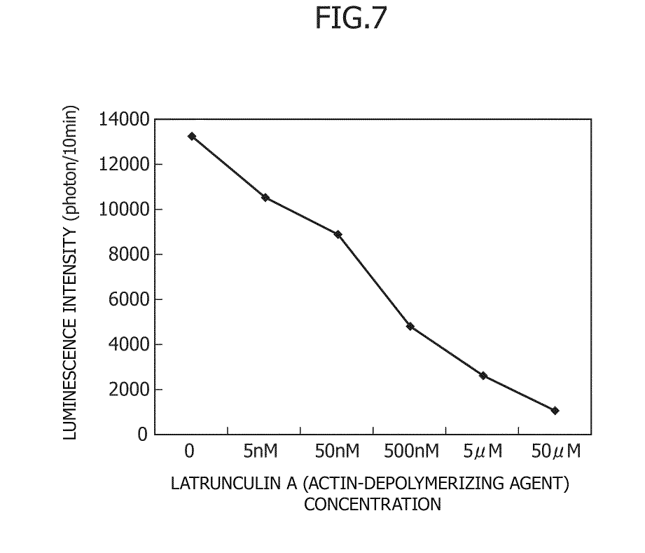

When varying concentrations of the actin polymerization inhibitor were administered, the luminescence was observed to be decreased in a concentration-dependent manner (FIG. 7). This is the evidence that the luminescence of the prepared protein serves as an index for actin polymerization.

### Example 9

HEK293T cells were treated with latrunculin A and then fixed in 4% paraformaldehyde, and only polymerized actin was stained using F-Actin Visualization Biochem Kit (Cosmo Bio Co., Ltd.).

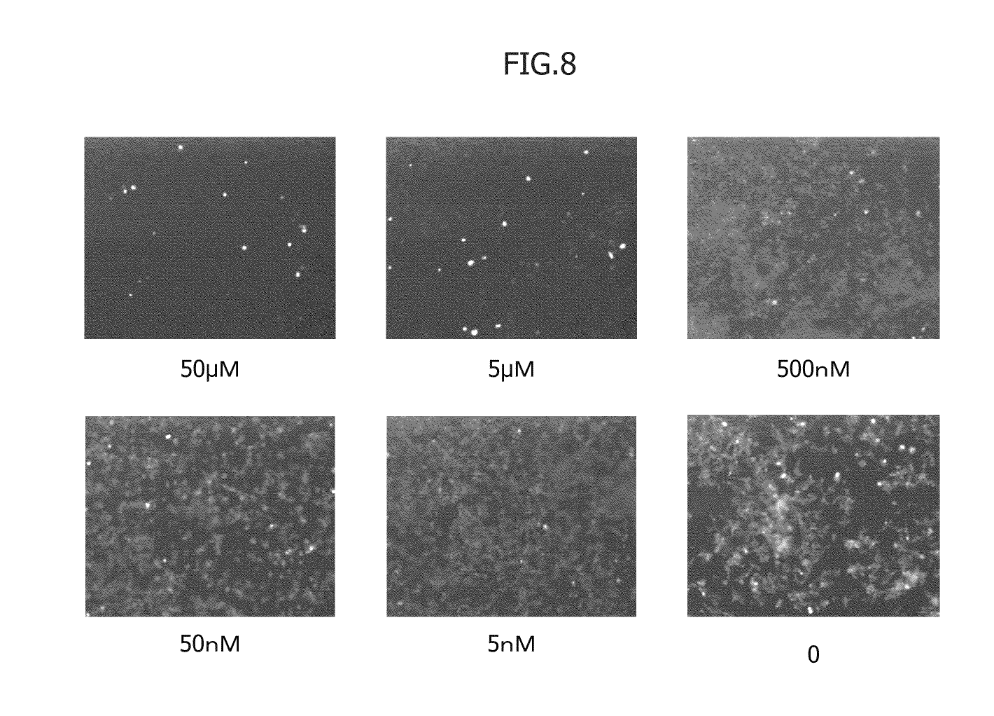

An image of polymerized actin stained with rhodamine-phalloidin in the presence of varying concentrations of the polymerization inhibitor was confirmed. The polymerized actin is decreased in a concentration-dependent manner (FIG. 8), as in the change in luminescence intensity.

### Example 10

Each sequence was inserted using restriction sites NheI, EcoRI, BamHI, and NotI of a pEGFP-N1 plasmid (Clontech Laboratories, Inc.). One of the sequences LucN-actin and LucC-actin was inserted between NheI and EcoRI. The other sequence was inserted between BamHI and NotI. An IRES sequence was inserted between EcoRI and BamHI.

Fusion protein 14: LucN-Actin-IRES-LucC-actin
Fusion protein 15: LucC-Actin-IRES-LucN-actin

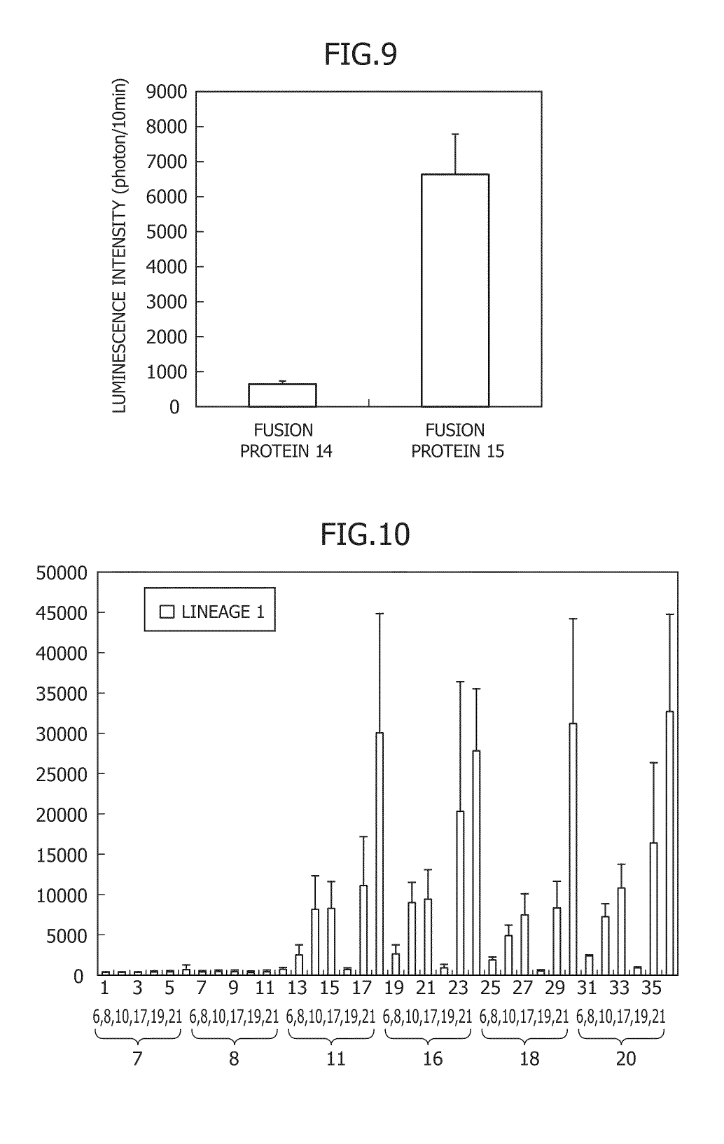

The luminescence intensity could be increased by exchanging the sequences located before and after IRES (FIG. 9).

### Example 11

### Plasmid Preparation for Probe Protein Screening for Measuring KID-KIX Binding in Two-Molecule-Type System

(1) Plasmids were constructed using a Multisite Gateway System to obtain fusion proteins having NLS, KID, KIX, the C-terminal fragment of luciferase (LucC), and the N-terminal fragment of luciferase (LucN) shown below. KGGRADPAFLYKVE (SEQ ID NO: 58) represents a sequence added by plasmid construction using a Multisite Gateway.

Fusion protein 22:
NLS-KID-KGGRADPAFLYKVE-LucN (NLS-KID-LucN)
Fusion protein 23:
NLS-KID-KGGRADPAFLYKVE-LucC (NLS-KID-LucC)
Fusion protein 24:
NLS-KIX-KGGRADPAFLYKVE-LucN (NLS-KIX-LucN)
Fusion protein 25:
NLS-KIX-KGGRADPAFLYKVE-LucC (NLS-KIX-LucC)
Fusion protein 26:
NLS-LucN-KGGRADPAFLYKVE-KID (NLS-LucN-KID)
Fusion protein 27:
NLS-LucC-KGGRADPAFLYKVE-KID (NLS-LucC-KID)
Fusion protein 28:
NLS-LucN-KGGRADPAFLYKVE-KIX (NLS-LucN-KIX)
Fusion protein 29:
NLS-LucC-KGGRADPAFLYKVE-KIX (NLS-LucC-KIX)

(2) A fusion protein 30 was prepared with the DNA sequence of the fusion protein 22 as a template using KOD plus a mutagenesis kit. Specifically, the plasmid for the fusion protein 22 was used as a template to perform PCR using a reverse primer comprising the KID tail-binding sequence plus a sequence encoding the first half of the linker sequence (added to the 5′ end of the binding sequence) 5′-CGCCCCCACTACCCCCACCTCCAGTCTCCTCTTCTGACTTTTCTTCT-3′ (SEQ ID NO: 43), and

a forward primer comprising the primer recognizing the head of LucN, plus a sequence encoding the last half of the linker sequence 5′-GAGGTAGCGGTGGCGGTGGTAGTATGGAAGATGCCAAAAACATTAAG-3′ ((SEQ ID NO: 44).
Subsequently, the template plasmid was degraded with an enzyme DpnI selectively digesting only methylated DNA. Furthermore, the PCR product was ligated at both the ends and cloned in E. coli to obtain the following fusion protein:

fusion protein 30: NLS-KID-GGGGSGGGGSGGGGS-LucN.

(3) A fusion protein 31 was prepared with the DNA sequence of the fusion protein 23 as a template using KOD plus mutagenesis kit. Specifically, the plasmid for the fusion protein 23 was used as a template to perform PCR using a reverse primer comprising the KID tail-binding sequence plus a sequence encoding the first half of the linker sequence (added to the 5′ end of the binding sequence) 5′-CGCCCCCACTACCCCCACCTCCAGTCTCCTCTTCTGACTTTTCTTCT-3′ (SEQ ID NO: 43), and

a forward primer comprising the sequence recognizing the head of LucC, plus a sequence encoding the last half of the linker sequence 5′-GAGGTAGCGGTGGCGGTGGTAGTGGCTGGCTGCACAGCGGCGACATCG-3′ (SEQ ID NO: 45).
Subsequently, the template plasmid was degraded with an enzyme DpnI selectively digesting only methylated DNA. Furthermore, the PCR product was ligated at both the ends and cloned in E. coli to obtain the following fusion protein:

fusion protein 31: NLS-KID-GGGGSGGGGSGGGGS-LucC.

(4) A fusion protein 32 was prepared with the DNA sequence of the fusion protein 24 as a template using KOD plus mutagenesis kit. Specifically, the plasmid for the fusion protein 24 was used as a template to perform PCR using a reverse primer comprising the KIX tail-binding primer plus a sequence encoding the first half of the linker sequence (added to the 5′ end of the binding sequence) 5′-CGCCCCCACTACCCCCACCTCCTTCTTCTAGTTCTTTTTGTATTTTA-3′ (SEQ ID NO: 46), and

a forward primer comprising the sequence recognizing the head of LucN, plus a sequence encoding the last half of the linker sequence 5′-GAGGTAGCGGTGGCGGTGGTAGTATGGAAGATGCCAAAAACATTAAG-3′ (SEQ ID NO: 47).
Subsequently, the template plasmid was degraded with an enzyme DpnI selectively digesting only methylated DNA. Furthermore, the PCR product was ligated at both ends and cloned in E. coli to obtain the following fusion protein:

fusion protein 32: NLS-KIX-GGGGSGGGGSGGGGS-LucN.

(5) A fusion protein 33 was prepared with the DNA sequence of the fusion protein 25 as a template using KOD plus mutagenesis kit. Specifically, the plasmid for the fusion protein 25 was used as a template to perform PCR using a reverse primer comprising the KIX tail-binding primer plus a sequence encoding the first half of the linker sequence (added to the 5′ end of the binding sequence) 5′-CGCCCCCACTACCCCCACCTCCTTCTTCTAGTTCTTTTTGTATTTTA-3′ (SEQ ID NO: 46), and

a forward primer comprising the sequence recognizing the head of LucC, plus a sequence encoding the last half of the linker sequence 5′-GAGGTAGCGGTGGCGGTGGTAGTGGCTGGCTGCACAGCGGCGACATCG-3′ (SEQ ID NO: 48).
Subsequently, the template plasmid was degraded with an enzyme DpnI selectively digesting only methylated DNA. Furthermore, the PCR product was ligated at both ends and cloned in E. coli to obtain the following fusion protein:

fusion protein 33: NLS-KIX-GGGGSGGGGSGGGGS-LucC.

(6) A fusion protein 34 was prepared with the DNA sequence of the fusion protein 26 as a template using KOD plus mutagenesis kit. Specifically, the plasmid for the fusion protein 26 was used as a template to perform PCR using a reverse primer comprising the LucN tail-binding primer plus a sequence encoding the first half (GGGGS) of a linker sequence (added to the 5′ end of the binding sequence) 5′-ACTACCCCCACCTCCGTCCTTGTCGATGAGAGCGTTTGTA-3′ (SEQ ID NO: 49), and

a primer comprising a sequence encoding the N-terminal region (KGGRADPA) of the linker in the fusion protein 26, plus a sequence encoding the last half (GGGGS) of the linker 5′-GGTGGCGGTGGTAGTAAGGGTGGGCGCGCCGAGCCAGCT-3′ (SEQ ID NO: 50).
Subsequently, the template plasmid was degraded with an enzyme DpnI selectively digesting only methylated DNA. Furthermore, the PCR product was ligated at both ends and cloned in E. coli to obtain the following fusion protein:

fusion protein 34:
NLS-LucN-GGGGSGGGGSKGGRADPAFLYKVE-KID.

(7) A fusion protein 35 was prepared with the DNA sequence of the fusion protein 27 as a template using KOD plus mutagenesis kit. Specifically, the plasmid for the fusion protein 27 was used as a template to perform PCR using a reverse primer comprising the LucC tail-binding primer plus a sequence encoding the first half (GGGGS) of a linker sequence (added to the 5′ end of the binding sequence) 5′-ACTACCCCCACCTCCCACGGCGATCTTGCCGCCCTTC-3′ (SEQ ID NO: 51), and

a primer comprising a sequence encoding the N-terminal region (KGGRADPA) of the linker in the fusion protein 27, plus a sequence encoding the last half (GGGGS) of the linker 5′-GGTGGCGGTGGTAGTAAGGGTGGGCGCGCCGAGCCAGCT-3′ (SEQ ID NO: 50).
Subsequently, the template plasmid was degraded with an enzyme DpnI selectively digesting only methylated DNA. Furthermore, the PCR product was ligated at both ends and cloned in E. coli to obtain the following fusion protein:

fusion protein 35:
NLS-LucC-GGGGSGGGGSKGGRADPAFLYKVE-KID.

(8) A fusion protein 36 was prepared with the DNA sequence of the fusion protein 28 as a template using KOD plus mutagenesis kit. Specifically, the plasmid for the fusion protein 28 was used as a template to perform PCR using a reverse primer comprising the LucN tail-binding primer plus a sequence encoding the first half (GGGGS) of a linker sequence (added to the 5′ end of the binding sequence) 5′-ACTACCCCCACCTCCGTCCTTGTCGATGAGAGCGTTTGTA-3′ (SEQ ID NO: 49), and

a primer comprising a sequence encoding the N-terminal region (KGGRADPA) of the linker in the fusion protein 28, plus a sequence encoding the last half (GGGGS) of the linker 5′-GGTGGCGGTGGTAGTAAGGGTGGGCGCGCCGAGCCAGCT-3′ (SEQ ID NO: 50).
Subsequently, the template plasmid was degraded with an enzyme DpnI selectively digesting only methylated DNA. Furthermore, the PCR product was ligated at both ends and cloned in E. coli to obtain the following fusion protein:

fusion protein 36:
NLS-LucN-GGGGSGGGGSKGGRADPAFLYKVE-KIX.

(9) A fusion protein 37 was prepared with the DNA sequence of the fusion protein 29 as a template using KOD plus mutagenesis kit. Specifically, the plasmid for the fusion protein 29 was used as a template to perform PCR using a reverse primer comprising the LucC tail-binding primer plus a sequence encoding the first half (GGGGS) of a linker sequence (added to the 5′ end of the binding sequence) 5′-ACTACCCCCACCTCCCACGGCGATCTTGCCGCCCTTC-3′ (SEQ ID NO: 51), and

a primer comprising a sequence encoding the N-terminal region (KGGRADPA) of the linker in the fusion protein 29, plus a sequence encoding the last half (GGGGS) of the linker 5′-GGTGGCGGTGGTAGTAAGGGTGGGCGCGCCGAGCCAGCT-3′ (SEQ ID NO: 50).
Subsequently, the template plasmid was degraded with an enzyme DpnI selectively digesting only methylated DNA. Furthermore, the PCR product was ligated at both ends and cloned in E. coli to obtain the following fusion protein:

fusion protein 37:
NLS-LucC-GGGGSGGGGSKGGRADPAFLYKVE-KIX.

(10) A fusion protein 38 was prepared with the DNA sequence of the fusion protein 26 as a template using KOD plus mutagenesis kit. Specifically, the plasmid for the fusion protein 26 was used as a template to perform PCR using the LucN tail-binding reverse primer 5′-GTCCTTGTCGATGAGAGCGTTTGTA-3′ (SEQ ID NO: 9), and

the forward primer binding to the first 24 bases of the KID sequence 5′-CAGATTTCAACTATTGCAGAAAGTG-3′ (SEQ ID NO: 15).
Subsequently, the template plasmid was degraded with an enzyme DpnI selectively digesting only methylated DNA. Furthermore, the PCR product was ligated at both ends and cloned in E. coli to obtain the following fusion protein:

fusion protein 38: NLS-LucN-KID.

(11) A fusion protein 39 was prepared with the DNA sequence of the fusion protein 27 as a template using KOD plus mutagenesis kit. Specifically, the plasmid for the fusion protein 27 was used as a template to perform PCR using the LucC tail-binding reverse primer 5′-CACGGCGATCTTGCCGCCCTTC-3′ (SEQ ID NO: 52), and

the forward primer binding to the first 24 bases of the KID sequence 5′-CAGATTTCAACTATTGCAGAAAGTG-3′ (SEQ ID NO: 15).
Subsequently, the template plasmid was degraded with an enzyme DpnI selectively digesting only methylated DNA. Furthermore, the PCR product was ligated at both ends and cloned in E. coli to obtain the following fusion protein:

fusion protein 39: NLS-LucC-KID.

(12) A fusion protein 40 was prepared with the DNA sequence of the fusion protein 29 as a template using KOD plus mutagenesis kit. Specifically, the plasmid for the fusion protein 29 was used as a template to perform PCR using the LucC tail-binding reverse primer 5′-CACGGCGATCTTGCCGCCCTTC-3′ (SEQ ID NO: 52), and

the forward primer binding to the first 24 bases of the KIX sequence 5′-GGTGTTCGAAAAGGCTGGCATGAAC-3′ (SEQ ID NO: 18).
Subsequently, the template plasmid was degraded with an enzyme DpnI selectively digesting only methylated DNA. Furthermore, the PCR product was ligated at both the ends and cloned in E. coli to obtain the following fusion protein:

fusion protein 40: NLS-LucC-KIX.

(13) A fusion protein 41 was prepared with the DNA sequence of the fusion protein 28 as a template using KOD plus mutagenesis kit. Specifically, the plasmid for the fusion protein 28 was used as a template to perform PCR using the LucN tail-binding reverse primer 5′-GTCCTTGTCGATGAGAGCGTTTGTA-3′ (SEQ ID NO: 9), and

the forward primer binding to the first 24 bases of the KIX sequence 5′-GGTGTTCGAAAAGGCTGGCATGAAC-3′ (SEQ ID NO: 18).
Subsequently, the template plasmid was degraded with an enzyme DpnI selectively digesting only methylated DNA. Furthermore, the PCR product was ligated at both ends and cloned in E. coli to obtain the following fusion protein:

fusion protein 41: NLS-LucN-KIX.

(14) A fusion protein 42 was prepared with the DNA sequence of the fusion protein 26 as a template using KOD plus mutagenesis kit. Specifically, the plasmid for the fusion protein 26 was used as a template to perform PCR using the reverse primer comprising the LucN tail-binding sequence plus a sequence encoding the first half of the linker sequence (added to the 5′ end of the binding sequence) 5′-CGCCCCCACTACCCCCACCTCCGTCCTTGTCGATGAGAGCGTTTGTA-3′ (SEQ ID NO: 33), and

a forward primer comprising the forward primer recognizing the KID head, plus a sequence encoding the last half of the linker sequence 5′-GAGGTAGCGGTGGCGGTGGTAGTCAGATTTCAACTATTGCAGAAAGTG-3′ (SEQ ID NO: 53).
Subsequently, the template plasmid was degraded with an enzyme DpnI selectively digesting only methylated DNA. Furthermore, the PCR product was ligated at both ends and cloned in E. coli to obtain the following fusion protein:

fusion protein 42: NLS-LucN-GGGGSGGGGSGGGGS-KID.

(15) A fusion protein 43 was prepared with the DNA sequence of the fusion protein 27 as a template using KOD plus mutagenesis kit. Specifically, the plasmid for the fusion protein 26 was used as a template to perform PCR using the reverse primer comprising the LucC tail-binding sequence plus a sequence encoding the first half of the linker sequence (added to the 5′ end of the binding sequence) 5′-CGCCCCCACTACCCCCACCTCCCACGGCGATCTTGCCGCCCTTC-3′ (SEQ ID NO: 54), and

a forward primer comprising the forward primer recognizing the KID head, plus a sequence encoding the last half of the linker sequence 5′-GAGGTAGCGGTGGCGGTGGTAGTCAGATTTCAACTATTGCAGAAAGTG-3′ (SEQ ID NO: 53).
Subsequently, the template plasmid was degraded with an enzyme DpnI selectively digesting only methylated DNA. Furthermore, the PCR product was ligated at both ends and cloned in E. coli to obtain the following fusion protein:

fusion protein 43: NLS-LucC-GGGGSGGGGSGGGGS-KID.

(16) A fusion protein 44 was prepared with the DNA sequence of the fusion protein 29 as a template using KOD plus mutagenesis kit. Specifically, the plasmid for the fusion protein 29 was used as a template to perform PCR using the reverse primer comprising the LucC tail-binding sequence plus a sequence encoding the first half of the linker sequence (added to the 5′ end of the binding sequence) 5′-CGCCCCCACTACCCCCACCTCCCACGGCGATCTTGCCGCCCTTC-3′ (SEQ ID NO: 54), and

a forward primer comprising the forward primer recognizing the KIX head, plus a sequence encoding the last half of the linker sequence 5′-GAGGTAGCGGTGGCGGTGGTAGTGGTGTTCGAAAAGGCTGGCATGAAC-3′ (SEQ ID NO: 55).
Subsequently, the template plasmid was degraded with an enzyme DpnI selectively digesting only methylated DNA. Furthermore, the PCR product was ligated at both ends and cloned in E. coli to obtain the following fusion protein:

fusion protein 44: NLS-LucC-GGGGSGGGGSGGGGS-KIX.

(17) A plasmid 45 was prepared with the DNA sequence of the fusion protein 28 as a template using KOD plus mutagenesis kit. Specifically, the plasmid for the fusion protein 28 was used as a template to perform PCR using the reverse primer comprising the LucN tail-binding sequence plus a sequence encoding the first half of the linker sequence (added to the 5′ end of the binding sequence) 5′-CGCCCCCACTACCCCCACCTCCGTCCTTGTCGATGAGAGCGTTTGTA-3′ (SEQ ID NO: 33), and

a forward primer comprising the forward primer recognizing the KIX head, plus a sequence encoding the last half of the linker sequence 5′-GAGGTAGCGGTGGCGGTGGTAGTGGTGTTCGAAAAGGCTGGCATGAAC-3′ (SEQ ID NO: 55).
Subsequently, the template plasmid was degraded with an enzyme DpnI selectively digesting only methylated DNA. Furthermore, the PCR product was ligated at both ends and cloned in E. coli to obtain the following fusion protein:

fusion protein 45: NLS-LucN-GGGGSGGGGSGGGGS-KIX.

(18) A fusion protein 46 was prepared with the DNA sequence of the fusion protein 31 as a template using KOD plus mutagenesis kit. The plasmid for the fusion protein was used as a template to perform PCR using a forward primer comprising a sequence binding to bases 96 to 120 of the KID sequence except that thymidine 97 was substituted by guanine 5′-TGCCTACAGGAAAATTTTGAATGAC-3′ (SEQ ID NO: 56), and

a reverse primer binding to a sequence of bases 71 to 95 thereof 5′-GGCCTCCTTGAAAGGATTTCCCTTC-3′ (SEQ ID NO: 57).
Subsequently, the template plasmid was degraded with an enzyme DpnI selectively digesting only methylated DNA. Furthermore, the PCR product was ligated at both ends and cloned in E. coli to obtain the following fusion protein:

fusion protein 46:
NLS-KID(S33A)-GGGGSGGGGSGGGGS-LucC.

### Example 12

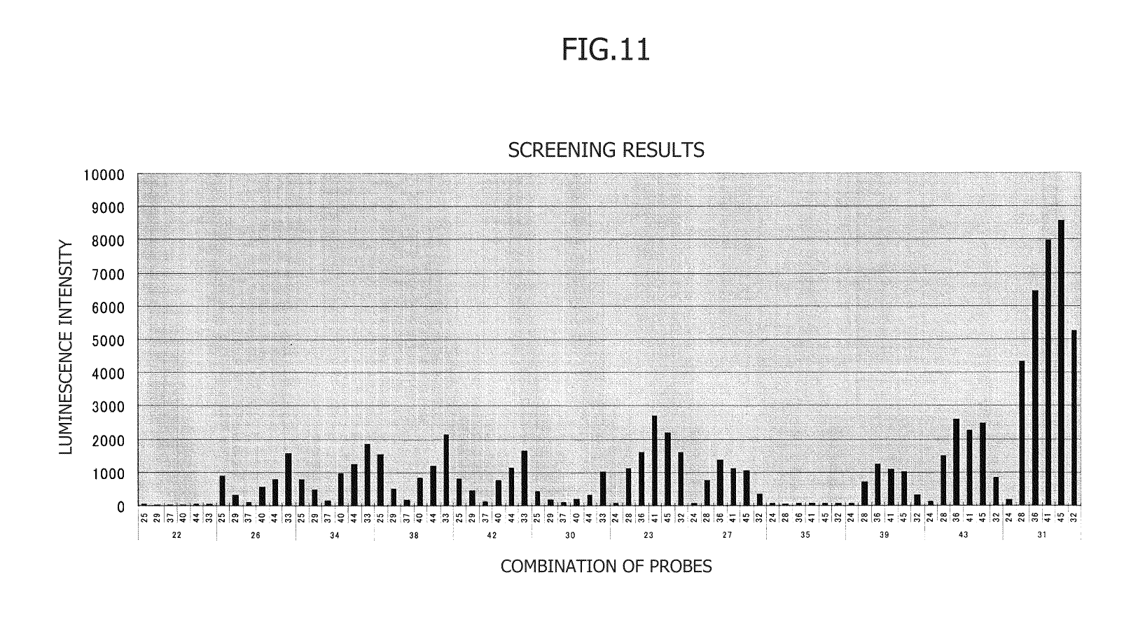

One each was selected from the plasmids for two groups of the fusion proteins (22, 26, 30, 34, 38, 42) and (25, 29, 33, 37, 40, 44). Combinations of the selected plasmids were gene-transferred to HEK293 cells, and the luminescence intensity (the number of photons observed per 10 minutes) was measured. Likewise, one each was selected from the plasmids for two groups of the fusion proteins (23, 27, 31, 35, 39, 43) and (24, 28, 32, 36, 41, 45) in combination, and the luminescence intensity was measured in the same way as above. The results are shown in FIG. 11. The abscissa represents combinations of the plasmids used in the gene transfer, and the ordinate represents the number of observed photons per 2-minute exposure time, i.e., luminescence intensity.

### Example 13

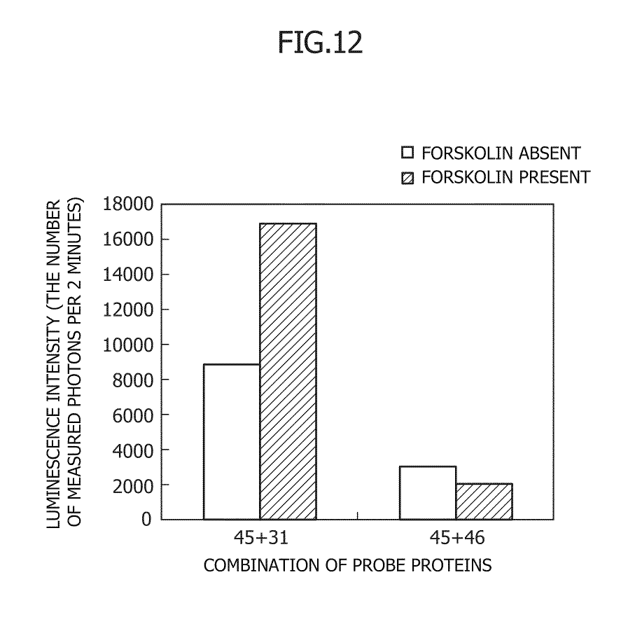

The combination of the fusion proteins 31 and 45 that exhibited the largest luminescence intensity in FIG. 11 was further analyzed. Thymidine 97 in the 180-bp DNA sequence of the KID sequence in the fusion protein 31 was substituted by guanine to prepare a fusion protein 46 comprising the amino acid sequence of the fusion protein 31 except that serine 33 was substituted by alanine. Combinations (31,45) and (46,45) were separately expressed in HEK293 cells. Forskolin was added thereto at a final concentration of 10 μM, and after 30 minutes, the luminescence was measured for 2 minutes. It was confirmed that forskolin increases the concentration of intracellular cAMP, which in turn activates PKA to phosphorylate serine 33 of KID, via which KID binds to the KIX domain. Moreover, in the combination (46,45), even the addition of forskolin does not increase luminescence, demonstrating that this probe protein specifically detects the phosphorylation of serine 33 in the KID domain (FIG. 12).

### Example 14

### Transgenic Mouse Preparation

Plasmid Construction

Plasmids for transgenic mouse preparation were prepared using a plasmid pCAGGS, which has: a hybrid Chicken b-Actin promoter/CMV (cytomegalovirus)-IE Enhancer (CAG) promoter; restriction sites in which a gene to be expressed can be inserted; and a rabbit beta-Globin poly-A signal added downstream of the gene. This plasmid also contains the coding region of an ampicillin resistance gene. This plasmid was the same as that reported in Journal of Biochemistry, 2003, vol. 133, p. 423-427. To this plasmid, a sequence encoding a fusion protein represented by the fusion protein 5 was inserted. The plasmid was further treated with restriction enzymes present at both the ends of the promoter+poly-A signal sequence to separate a region containing the promoter, the gene to be expressed, and the poly-A signal from a region containing the ampicillin resistance gene. The region containing the promoter, the gene to be expressed, and the poly-A signal was separated and purified by agarose gel electrophoresis, filtered through a 0.22-μm filter to 2.5 ng/μl, and finally used in microinjection.

Pronuclear Stage Embryo Collection and Microinjection

For artificial insemination, sperm cells were collected from a male mouse (C57BL/6J, 10-week-old) and precultured.

Likewise, for artificial insemination, eggs were collected from a female mouse (C57BL/6J, 10-week-old) that received superovulation treatment (intraperitoneal administration of PMSG and hCG at 5 IU at 48-hour intervals), and precultured. The sperm cells were added to the culture solution containing the eggs to perform external fertilization. Five to six hours later, the fertilized eggs were washed and screened for those confirmed to have the pronuclei. The prepared DNA was microinjected into the male pronuclei of the fertilized eggs and cultured until the next day. Normally developing fertilized eggs were picked up and transplanted to the uterine tube of a pseudopregnant female mouse (ICR, 10-weeks-old). The tails of the obtained newborns were used to confirm the presence of a transgenic mouse having the inserted gene by PCR and southern blotting.

The transgenic mouse having the inserted gene can be used, for example, in maze learning, to observe the identification of nerve cells activated by memory formation, the timing and intensity of the activation, etc., based on the luminescence of the luciferase. Moreover, a drug promoting or inhibiting memory formation can be screened by administering a drug to the transgenic mouse having the inserted gene.

### INDUSTRIAL APPLICABILITY

According to the present invention, neural activity such as memory formation or sensation in live animals can be measured in real time. Specifically, the kinetics of CREB or actin closely related to brain functions can be measured in real time. Thus, for example, for the screening of a drug controlling brain functions, the influence of a certain drug on the activity of the particular protein such as CREB or actin can be measured continually over a long period in the same animal. Moreover, when and where the target protein is activated during the formation of memory learning can be examined over a long period in the same animal. This helps elucidate the mechanism of memory learning.

Moreover, actin is polymerized during cell division to form a contractile ring. An active site of cell division (growing site, tumor, or cancer tissue) can also be identified in animals with the nuclear genome encoding the probe sequence of the present invention.

1. A probe for visualizing neural activity, the probe consisting of one or two molecule(s), wherein the probe is selected from any one or more of:
(1) a probe consisting of one molecule selected from a fusion protein 1 (NLS-LucN-KID-GGGGSGGGGSGGGGS-KIX-LucC) and a fusion protein 5 (NLS-LucN-KID-LucC);
(2) a probe consisting of two molecules selected from
a combination of a fusion protein 26 (NLS-LucN-KGGRADPAFLYKVE-KID) and a fusion protein 33 (NLS-KIX-GGGGSGGGGSGGGGS-LucC),
a combination of a fusion protein 34 (NLS-LucN-GGGGSGGGGSKGGRADPAFLYKVE-KID) and the fusion protein 33, a combination of the fusion protein 34 and a fusion protein 44 (NLS-LucC-GGGGSGGGGSGGGGS-KIX),
a combination of a fusion protein 38 (NLS-LucN-KID) and a fusion protein 25 (NLS-KIX-KGGRADPAFLYKVE-LucC), a combination of the fusion protein 38 and the fusion protein 33, a combination of the fusion protein 38 and the fusion protein 44,
a combination of a fusion protein 42 (NLS-LucN-GGGGSGGGGSGGGGS-KID) and the fusion protein 33, a combination of the fusion protein 42 and the fusion protein 44,
a combination of a fusion protein 30 (NLS-KID-GGGGSGGGGSGGGGS-LucN) and the fusion protein 33,
a combination of a fusion protein 23 (NLS-KID-KGGRADPAFLYKVE-LucC) and a fusion protein 28 (NLS-LucN-KGGRADPAFLYKVE-KIX), a combination of the fusion protein 23 and a fusion protein 32 (NLS-KIX-GGGGSGGGGSGGGGS-LucN), a combination of the fusion protein 23 and a fusion protein 36 (NLS-LucN-GGGGSGGGGSKGGRADPAFLYKVE-KIX), a combination of the fusion protein 23 and a fusion protein 41 (NLS-LucN-KIX), a combination of the fusion protein 23 and a fusion protein 45,
a combination of a fusion protein 27 (NLS-LucC-KGGRADPAFLYKVE-KID) and the fusion protein 36, a combination of the fusion protein 27 and the fusion protein 41, a combination of the fusion protein 27 and the fusion protein 45,
a combination of a fusion protein 39 (NLS-LucC-KID) and the fusion protein 36, a combination of the fusion protein 39 and the fusion protein 41, a combination of the fusion protein 39 and the fusion protein 45,
a combination of a fusion protein 43 (NLS-LucC-GGGGSGGGGSGGGGS-KID) and the fusion protein 28, a combination of the fusion protein 43 and the fusion protein 36, a combination of the fusion protein 43 and the fusion protein 41, a combination of the fusion protein 43 and the fusion protein 45,
a combination of a fusion protein 31 (NLS-KID-GGGGSGGGGSGGGGS-LucC) and the fusion protein 28, a combination of the fusion protein 31 and the fusion protein 36, a combination of the fusion protein 31 and the fusion protein 41, a combination of the fusion protein 31 and the fusion protein 45, and a combination of the fusion protein 31 and the fusion protein 32; and
(3) a probe consisting of two molecules selected from
a combination of a fusion protein 11 (LucN-KGGRADPAFLYKVE-Actin) and a fusion protein 8 (Actin-GGGGSGGGGSGGGGS-LucC), a combination of the fusion protein 11 and a fusion protein 10 (LucC-KGGRADPAFLYKVE-Actin), a combination of the fusion protein 11 and a fusion protein 19 (Actin-LucC), a combination of the fusion protein 11 and a fusion protein 21 (Actin-GGGGSGGGGSGGGGS-LucC),
a combination of a fusion protein 16 (Actin-GGGGSGGGGSKGGRADPAFLYKVE-LucN) and the fusion protein 8, a combination of the fusion protein 16 and the fusion protein 10, a combination of the fusion protein 16 and the fusion protein 19, a combination of the fusion protein 16 and the fusion protein 21,
a combination of a fusion protein 18 (Actin-LucN) and the fusion protein 8, a combination of the fusion protein 18 and the fusion protein 10, a combination of the fusion protein 18 and the fusion protein 19, a combination of the fusion protein 18 and the fusion protein 21,
a combination of a fusion protein 20 (Actin-GGGGSGGGGSGGGGS-LucN) and the fusion protein 8, a combination of the fusion protein 20 and the fusion protein 10, a combination of the fusion protein 20 and the fusion protein 19, and a combination of the fusion protein 20 and the fusion protein 21.

2. The probe according to claim 1, wherein the probe is selected from any one or more of:
(1) a probe consisting of one molecule selected from the fusion protein 1 and the fusion protein 5;
(2) a probe consisting of two molecules selected from
the combination of the fusion protein 31 and the fusion protein 28, the combination of the fusion protein 31 and the fusion protein 36, the combination of the fusion protein 31 and the fusion protein 41, the combination of the fusion protein 31 and the fusion protein 45, and the combination of the fusion protein 31 and the fusion protein 32; and
(3) a probe consisting of two molecules selected from
the combination of the fusion protein 11 and the fusion protein 10, the combination of the fusion protein 11 and the fusion protein 21, the combination of the fusion protein 16 and the fusion protein 19, the combination of the fusion protein 16 and the fusion protein 21, the combination of the fusion protein 18 and the fusion protein 21, and the combination of the fusion protein 20 and the fusion protein 21.

3. (canceled)

4. (canceled)

5. A DNA encoding a probe for visualizing neural activity, wherein the DNA is selected from any of:
(1) a DNA comprising a sequence encoding a fusion protein 1, or a DNA comprising a sequence encoding a fusion protein 5;
(2) a DNA comprising a sequence encoding a fusion protein 26 and a sequence encoding a fusion protein 33,
a DNA comprising a sequence encoding a fusion protein 34 and the sequence encoding the fusion protein 33, a DNA comprising the sequence encoding the fusion protein 34 and a sequence encoding a fusion protein 44,
a DNA comprising a sequence encoding a fusion protein 38 and a sequence encoding a fusion protein 25, a DNA comprising the sequence encoding the fusion protein 38 and the sequence encoding the fusion protein 33, a DNA comprising the sequence encoding the fusion protein 38 and the sequence encoding the fusion protein 44,
a DNA comprising a sequence encoding a fusion protein 42 and the sequence encoding the fusion protein 33, a DNA comprising the sequence encoding the fusion protein 42 and the sequence encoding the fusion protein 44,
a DNA comprising a sequence encoding a fusion protein 30 and the sequence encoding the fusion protein 33,
a DNA comprising a sequence encoding a fusion protein 23 and a sequence encoding a fusion protein 28, a DNA comprising the sequence encoding the fusion protein 23 and a sequence encoding a fusion protein 32, a DNA comprising the sequence encoding the fusion protein 23 and a sequence encoding a fusion protein 36, a DNA comprising the sequence encoding the fusion protein 23 and a sequence encoding a fusion protein 41, a DNA comprising the sequence encoding the fusion protein 23 and a sequence encoding a fusion protein 45,
a DNA comprising a sequence encoding a fusion protein 27 and the sequence encoding the fusion protein 36, a DNA comprising the sequence encoding the fusion protein 27 and the sequence encoding the fusion protein 41, a DNA comprising the sequence encoding the fusion protein 27 and the sequence encoding the fusion protein 45,
a DNA comprising a sequence encoding a fusion protein 39 and the sequence encoding the fusion protein 36, a DNA comprising the sequence encoding the fusion protein 39 and the sequence encoding the fusion protein 41, a DNA comprising the sequence encoding the fusion protein 39 and the sequence encoding the fusion protein 45;
a DNA comprising a sequence encoding a fusion protein 43 and the sequence encoding the fusion protein 28, a DNA comprising the sequence encoding the fusion protein 43 and the sequence encoding the fusion protein 36, a DNA comprising the sequence encoding the fusion protein 43 and the sequence encoding the fusion protein 41, a DNA comprising the sequence encoding the fusion protein 43 and the sequence encoding the fusion protein 45;
a DNA comprising a sequence encoding a fusion protein 31 and the sequence encoding the fusion protein 28, a DNA comprising the sequence encoding the fusion protein 31 and the sequence encoding the fusion protein 36, a DNA comprising the sequence encoding the fusion protein 31 and the sequence encoding the fusion protein 41, a DNA comprising the sequence encoding the fusion protein 31 and the sequence encoding the fusion protein 45, or a DNA comprising the sequence encoding the fusion protein 31 and the sequence encoding the fusion protein 32; and
(3) a DNA comprising a sequence encoding a fusion protein 11 and a sequence encoding a fusion protein 8, a DNA comprising the sequence encoding the fusion protein 11 and a sequence encoding a fusion protein 10, a DNA comprising the sequence encoding the fusion protein 11 and a sequence encoding a fusion protein 19, a DNA comprising the sequence encoding the fusion protein 11 and a sequence encoding a fusion protein 21,
a DNA comprising a sequence encoding a fusion protein 16 and the sequence encoding the fusion protein 8, a DNA comprising the sequence encoding the fusion protein 16 and the sequence encoding the fusion protein 10, a DNA comprising the sequence encoding the fusion protein 16 and the sequence encoding the fusion protein 19, a DNA comprising the sequence encoding the fusion protein 16 and the sequence encoding the fusion protein 21,
a DNA comprising a sequence encoding a fusion protein 18 and the sequence encoding the fusion protein 8, a DNA comprising the sequence encoding the fusion protein 18 and the sequence encoding the fusion protein 10, a DNA comprising the sequence encoding the fusion protein 18 and the sequence encoding the fusion protein 19, a DNA comprising the sequence encoding the fusion protein 18 and the sequence encoding the fusion protein 21,
a DNA comprising a sequence encoding a fusion protein 20 and the sequence encoding the fusion protein 8, a DNA comprising the sequence encoding the fusion protein 20 and the sequence encoding the fusion protein 10, a DNA comprising the sequence encoding the fusion protein 20 and the sequence encoding the fusion protein 19, or a DNA comprising the sequence encoding the fusion protein 20 and the sequence encoding the fusion protein 21.

6. The DNA according to claim 5, wherein the DNA is selected from any of:
(1) the DNA comprising the sequence encoding the fusion protein 1, or the DNA comprising the sequence encoding the fusion protein 5;
(2) the DNA comprising the sequence encoding the fusion protein 31 and the sequence encoding the fusion protein 28, the DNA comprising the sequence encoding the fusion protein 31 and the sequence encoding the fusion protein 36, the DNA comprising the sequence encoding the fusion protein 31 and the sequence encoding the fusion protein 41, the DNA comprising the sequence encoding the fusion protein 31 and the sequence encoding the fusion protein 45, or the DNA comprising the sequence encoding the fusion protein 31 and the sequence encoding the fusion protein 32; and
(3) the DNA comprising the sequence encoding the fusion protein 11 and the sequence encoding the fusion protein 10, the DNA comprising the sequence encoding the fusion protein 11 and the sequence encoding the fusion protein 21, the DNA comprising the sequence encoding the fusion protein 16 and the sequence encoding the fusion protein 19, the DNA comprising the sequence encoding the fusion protein 16 and the sequence encoding the fusion protein 21, the DNA comprising the sequence encoding the fusion protein 18 and the sequence encoding the fusion protein 21, or the DNA comprising the sequence encoding the fusion protein 20 and the sequence encoding the fusion protein 21.

7. (canceled)

8. (canceled)

9. A method for visualizing neural activity, comprising the steps of:
producing a probe according to claim 1 in a nerve cell; and
measuring luminescence of the luciferase.

10. (canceled)

11. (canceled)

12. (canceled)

13. A rodent transfected with a DNA according to claim 5.

14. A method for screening for a substance promoting neural activity of memory formation, the method using a rodent transfected with a DNA according to claim 5.
